(19) United States
(12) Patent Application Publication
(10) Pub. No.: US 2004/0265916 A1
(43) Pub. Date: Dec. 30, 2004

(54) Method for detecting selective androgen receptor modulators

(76) Inventors: James T. Dalton, Upper Arlington, OH (US); Duane D. Miller, Germantown, TN (US); Karen A. Veverka, Cordova, TN (US)

Correspondence Address: EITAN, PEARL, LATZER & COHEN ZEDEK LLP, 10 ROCKEFELLER PLAZA, SUITE 1001, NEW YORK, NY, 10020, US

(21) Appl. No.: 10/800,021

(22) Filed: Mar. 15, 2004

Related U.S. Application Data
(63) Continuation-in-part of application No. 10/270,232, filed on Oct. 15, 2002.
(63) Continuation-in-part of application No. 10/270,233, filed on Oct. 15, 2002.

(51) Int. Cl. G01N 33/53; G01N 33/567

(52) U.S. Cl. 435/7.2

(57) ABSTRACT

The present invention relates to analytical methods for detecting, identifying characterizing, purifying, obtaining structural information on, qualifying, screening, separating, fractionating and/or quantifying a Selective Androgen Receptor Modulator (SARM) compound in a sample, by obtaining a sample, and detecting the presence and/or concentration of a SARM compound in the sample. The methods of the present invention are highly robust, sensitive, specific, reliable, validated, validatable and reproducible assays that are of great commercial potential and value. The SARM compounds, either alone or as a composition, are useful for a) male contraception; b) treatment of a variety of hormone-related conditions, for example conditions associated with Androgen Decline in Aging Male (ADAM), such as fatigue, depression, decreased libido, sexual dysfunction, erectile dysfunction, hypogonadism, osteoporosis, hair loss, anemia, obesity, sarcopenia, osteopenia, osteoporosis, benign prostate hyperplasia, alterations in mood and cognition and prostate cancer; c) treatment of conditions associated with Androgen Decline in Female (ADIF), such as sexual dysfunction, decreased sexual libido, hypogonadism, sarcopenia, osteopenia, osteoporosis, alterations in cognition and mood, depression, anemia, hair loss, obesity, endometriosis, breast cancer, uterine cancer and ovarian cancer; d) treatment and/or prevention of acute and/or chronic muscular wasting conditions; e) preventing and/or treating dry eye conditions; f) oral androgen replacement therapy; and/or g) decreasing the incidence of, halting or causing a regression of prostate cancer. The development of a rapid, specific and sensitive assay for detection, identification and quantification of SARMs is of great value, allows better clinical evaluation and practice for a patient, is useful to monitor and reduce incidence of off-label use by athletes and others, offers important tools to investigate forensic and overdose situations, is useful in evalulating black market and bootleg anabolic compounds that are produced illegally, and can be used for disqualification of performers from competition when appropriate.

### CROSS-REFERENCE TO RELATED APPLICATIONS

This Application is a Continuation-in-Part Application of U.S. Ser. No. 10/270,232, filed Oct. 15, 2002, and of U.S. Ser. No. 10/270,233, Oct. 15, 2002, which are hereby incorporated by reference.

### FIELD OF INVENTION

The present invention relates to analytical methods for detecting, identifying characterizing, purifying, obtaining structural information on, qualifying, screening, separating, fractionating and/or quantifying Selective Androgen Receptor Modulator (SARM) compounds in a sample, for example a biological sample. SARM compounds are a novel class of androgen receptor targeting agents (ARTA), which demonstrate androgenic and anabolic activity of a nonsteroidal ligand for the androgen receptor, and which are useful for a) male contraception; b) treatment of a variety of hormone-related conditions, for example conditions associated with Androgen Decline in Aging Male (ADAM); c) treatment of conditions associated with Androgen Decline in Female (ADIF); d) treatment and/or prevention of acute and/or chronic muscular wasting conditions; e) preventing and/or treating dry eye conditions; f) oral androgen replacement therapy; and/or g) decreasing the incidence of, halting or causing a regression of prostate cancer.

### BACKGROUND OF THE INVENTION

The androgen receptor (“AR”) is a ligand-activated transcriptional regulatory protein that mediates induction of male sexual development and function through its activity with endogenous androgens. Androgens are generally known as the male sex hormones. The androgenic hormones are steroids which are produced in the body by the testes and the cortex of the adrenal gland or can be synthesized in the laboratory. Androgenic steroids play an important role in many physiologic processes, including the development and maintenance of male sexual characteristics such as muscle and bone mass, prostate growth, spermatogenesis, and the male hair pattern (Matsumoto, Endocrinol. Met. Clin. N. Am. 23:857-75 (1994)). The endogenous steroidal androgens include testosterone and dihydrotestosterone (“DHT”). Testosterone is the principal steroid secreted by the testes and is the primary circulating androgen found in the plasma of males. Testosterone is converted to DHT by the enzyme 5 alpha-reductase in many peripheral tissues. DHT is thus thought to serve as the intracellular mediator for most androgen actions (Zhou, et al., Molec. Endocrinol. 9:208-18 (1995)). Other steroidal androgens include esters of testosterone, such as the cypionate, propionate, phenylpropionate, cyclopentylpropionate, isocarporate, enanthate, and decanoate esters, and other synthetic androgens such as 7-Methyl-Nortestosterone (“MENT”) and its acetate ester (Sundaram et al., “7 Alpha-Methyl-Nortestosterone(MENT): The Optimal Androgen For Male Contraception,” Ann. Med., 25:199-205 (1993) (“Sundaram”)). Because the AR is involved in male sexual development and function, the AR is a likely target for effecting male contraception or other forms of hormone replacement therapy.

The androgen receptor is involved in many key physiologic processes. Accordingly, its modulation is of major clinical and biological significance, for example in contraception and in treatment of a variety of hormone-related conditions including conditions associated with Androgen Decline in Aging Male (ADAM), conditions associated with Androgen Decline in Female (ADIF), and prostate cancer.

Recently, a new class of nonsteroidal Androgen Receptor Targeting Agents (“ARTA”) has been discovered. These agents define a new subclass of Selective Androgen Receptor Modulator (SARM) compounds, with potent and tissue-selective in vivo pharmacologic activity (U.S. Pat. No. 6,492,554; Yin et al., Journal of Pharmacology and Experimental Therapeutics, in press, 2003). Several appropriately substituted SARM compounds have been shown to have an unexpected selective in-vivo androgenic and anabolic activity of a nonsteroidal ligand for the androgen receptor (AR).

These SARM compounds have been shown to be useful for a) male contraception; b) treatment of a variety of hormone-related conditions, for example conditions associated with Androgen Decline in Aging Male (ADAM), such as fatigue, depression, decreased libido, sexual dysfunction, erectile dysfunction, hypogonadism, osteoporosis, hair loss, anemia, obesity, sarcopenia, osteopenia, osteoporosis, benign prostate hyperplasia, alterations in mood and cognition and prostate cancer; c) treatment of conditions associated with ADIF, such as sexual dysfunction, decreased sexual libido, hypogonadism, sarcopenia, osteopenia, osteoporosis, alterations in cognition and mood, depression, anemia, hair loss, obesity, endometriosis, breast cancer, uterine cancer and ovarian cancer; d) treatment and/or prevention of acute and/or chronic muscular wasting conditions; e) preventing and/or treating dry eye conditions; f) oral androgen replacement therapy; and/or g) decreasing the incidence of, halting or causing a regession of prostate cancer.

The potent anabolic activity of Selective Androgen Receptor Modulators gives rise to the possibility of their abuse in athletes and in other populations. The development of a rapid, specific and sensitive assay for detection, identification and quantification of SARMs will allow better clinical evaluation and practice for a patient, used to monitor and reduce incidence of off-label use by athletes and others, offer important tools to investigate forensic and overdose situations, evalulate black market and bootleg anabolic compounds that are produced illegally, and can be used for disqualification of performers from competition when appropriate. The development of assays for detection, identification and quantification of SARMs thus has large commercial potential and is therefore highly desirable and urgently needed in the art.

### SUMMARY OF THE INVENTION

The present invention relates to analytical methods for detecting, identifying characterizing, purifying, obtaining structural information on, qualifying, screening, separating, fractionating and/or quantifying a Selective Androgen Receptor Modulator (SARM) compound in a sample. The methods of the present invention comprise obtaining a sample, such as a biological sample, and detecting the presence and/or concentration of a SARM compound in the sample. As demonstrated herein, the methods of the present invention are highly robust, sensitive, specific, reliable, validated, validatable and reproducible assays that are of great commercial potential and value.

Selective Androgen Receptor Modulator Compounds are a class of androgen receptor targeting agents (ARTA). Several of the SARM compounds have been found to have an unexpected androgenic and anabolic activity of a nonsteroidal ligand for the androgen receptor. Other SARM compounds have been found to have an unexpected antiandrogenic activity of a nonsteroidal ligand for the androgen receptor. The SARM compounds, either alone or as a composition, are useful for a) male contraception; b) treatment of a variety of hormone-related conditions, for example conditions associated with Androgen Decline in Aging Male (ADAM), such as fatigue, depression, decreased libido, sexual dysfunction, erectile dysfunction, hypogonadism, osteoporosis, hair loss, anemia, obesity, sarcopenia, osteopenia, osteoporosis, benign prostate hyperplasia, alterations in mood and cognition and prostate cancer; c) treatment of conditions associated with Androgen Decline in Female (ADIF), such as sexual dysfunction, decreased sexual libido, hypogonadism, sarcopenia, osteopenia, osteoporosis, alterations in cognition and mood, depression, anemia, hair loss, obesity, endometriosis, breast cancer, uterine cancer and ovarian cancer; d) treatment and/or prevention of acute and/or chronic muscular wasting conditions; e) preventing and/or treating dry eye conditions; f) oral androgen replacement therapy; and/or g) decreasing the incidence of, halting or causing a regression of prostate cancer.

The methods of the present invention comprise detecting the presence and/or concentration of a SARM compound in the sample. In one embodiment, the SARM compound is detected by subjecting an aliquot from the sample to any one or more of the following analytical detection methods: mass spectroscopy (MS), MS-MS, UV, IR, NMR, fluorescence, radiochemical detection, electrochemical detection, chemiluminscent detection, evaporative light scatter detection (ESLD), hyphenated techniques or methods, or any combination thereof. In one particular embodiment, the SARM compound is detected by measuring the UV absorbance of the SARM compound in the sample. In another particular embodiment, the SARM compound is detected by measuring the molecular ion peak of the SARM compound by mass spectrometry, for example by negative ion mass spectrometry.

In one embodiment, the method of the present invention further comprises quantifying the amount of SARM compound in the sample, i.e. determining the concentration of the SARM in the sample, by comparing the amount obtained from the sample with a reference sample comprising known amounts of a reference SARM compound.

In one embodiment, the method of the present invention further comprises subjecting the sample to a chromatographic separation prior to said detection step. In one embodiment, the chromatographic separation is by liquid chromatography (LC). In another embodiment, the chromatographic separation is by High Performance Liquid Chromatography (HPLC). In another embodiment, the chromatographic separation is by Thin Layer Chromatography (TLC). In another embodiment, the chromatographic separation is by capillary electrophoresis (CE). In another embodiment, the chromatographic separation is by microLC electrophoresis. In another embodiment, the chromatographic separation is by nano LC electrophoresis. In another embodiment, the chromatographic separation is by gel electrophoresis (GE). In another embodiment, the chromatographic separation is by isoelectric focusing gel electrophoresis. In another embodiment, the chromatographic separation is by sample concentration.

In one embodiment, the method of the present invention comprises separating the sample by chromatographic separation using any one of the detection methods described hereinabove, followed by a detection step using any one of the separation techniques described hereinabove. For example, in another embodiment, the method of the present invention comprises subjecting the sample to liquid chromatography (LC); and detecting the presence and/or concentration of a SARM compound in the sample by mass spectrometry (MS). In accordance with this embodiment, the method is a Liquid Chromatography/Mass Spectrometry method (LC/MS). In another embodiment, the mass spectrometry is negative ion mass spectrometry.

In another embodiment, the method of the present invention comprises subjecting the sample to High Performance Liquid Chromatography (HPLC); and detecting the presence and/or concentration of a SARM compound in the sample by measuring the UV absorbance of the SARM compound. In accordance with this embodiment, the method is a High Performance Liquid Chromatography/UV method (HPLC/UV).

In another embodiment, the method of the present invention comprises subjecting the sample to liquid chromatography (LC); and detecting the presence and/or concentration of a SARM compound in the sample by MS-MS. In accordance with this embodiment, the method is a LC/MS-MS method.

In another embodiment, the method of the present invention comprises subjecting the sample to capillary electrophoresis (CE); and detecting the presence and/or concentration of a SARM compound in the sample by mass spectrometry (MS). In accordance with this embodiment, the method is a CE/MS method.

It is apparent to a person skilled in the art that any other combination of separation/detection techniques is applicable, and is within the broad scope of the present invention.

In one embodiment, the chromatography step comprises producing a chromatograph of the sample, the chromatograph comprising a series of peaks representing individual chemical compounds contained in the sample. The individual chemical compounds are automatically collected into separate tube, and the content of each tube is analyzed by any of the detection methods outlined above. In another embodiment, the method further comprises the step of comparing output from the detection analysis for each tube with data identifying known SARM compounds expected to be present in the sample. In a particular embodiment, the data is stored in a database of a digital signal processor.

In one embodiment, the sample is a biological sample. In another embodiment, the sample is a blood serum sample, a plasma sample, a urine sample, a CSF sample, a saliva sample, a fecal sample, an isolated or precipitated fraction, a protein adduct, or a protein extract.

In one embodiment, the SARM is a compound represented by the structure of formula I, and/or an analog, derivative, metabolite, isomer, pharmaceutically acceptable salt, pharmaceutical product, hydrate, N-oxide, impurity, prodrug, polymorph, crystal, or any combination thereof

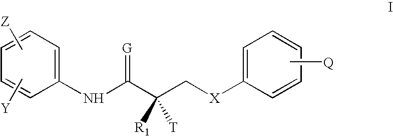

whereinG is O or S;

X is a bond, O, CH₂, NH, Se, PR, NO or NR;

T is OH, OR, —NHCOCH₃, or NHCOR

Z is NO₂, CN, COOH, COR, NHCOR or CONHR;

Y is CF₃, F, I, Br, Cl, CN, CR₃ or SnR₃;

Q is alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, SCN, NCS, OCN, NCO; or Q together with the benzene ring to which it is attached is a fused-ring system represented by structure A, B or C:

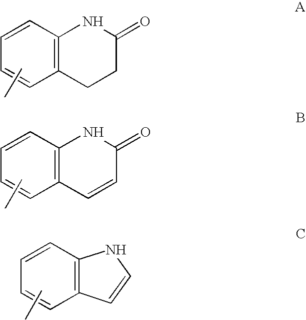

R is alkyl, haloalkyl, dihaloalkyl, trihaloalkyl, CH₂F, CHF₂, CF₃, CF₂CF₃, aryl, phenyl, halogen, alkenyl or OH; and

R₁ is CH₃, CH₂F, CHF₂, CF₃, CH₂CH₃, or CF₂CF₃.

In another embodiment, the SARM is a compound represented by the structure of formula II and/or an analog, derivative, metabolite, isomer, pharmaceutically acceptable salt, pharmaceutical product, hydrate, N-oxide, impurity, prodrug, polymorph, crystal, or any combination thereof

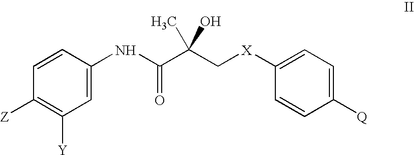

wherein

X is a bond, O, CH₂NH, Se, PR, NO or NR;

Z is NO₂, CN, COOH, COR, NHCOR or CONHR;

Y is CF₃, F, I, Br, Cl, CN, CR₃ or SnR₃;

Q is alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, SCN, NCS, OCN, NCO; or Q together with the benzene ring to which it is attached is a fused ring system represented by structure A, B or C:

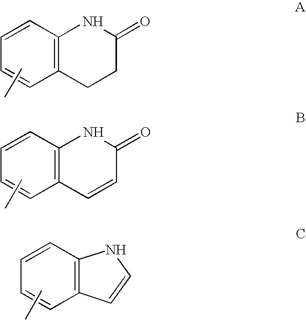

R is alkyl, haloalkyl, dihaloalkyl, trihaloalkyl, CH₂F, CHF₂, CF₃, CF₂CF₃, aryl, phenyl, halogen, alkenyl or OH.

In another embodiment, the SARM is a compound represented by the structure of formula III and/or an analog, derivative, metabolite, isomer, pharmaceutically acceptable salt, pharmaceutical product, hydrate, N-oxide, impurity, prodrug, polymorph, crystal, or any combination thereof

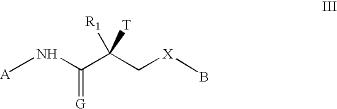

wherein X is a bond, O, CH₂, NH, Se, PR, NO or NR;

G is O or S;

R₁ is CH₃, CH₂F, CHF₂, CF₃, CH₂CH₃, or CF₂CF₃;

T is OH, OR, —NHCOCH₃, or NHCOR;

R is alkyl, haloalkyl, dihaloalkyl, trihaloalkyl, CH₂F, CHF₂, CF₃, CF₂CF₃, aryl, phenyl, halogen, alkenyl or OH;

A is a ring selected from:

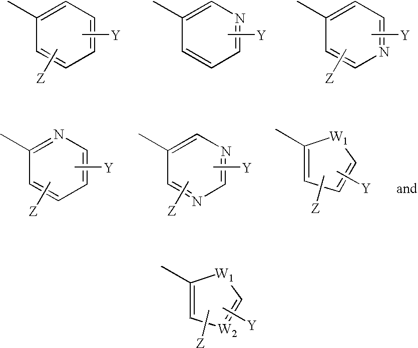

B is a ring selected from:

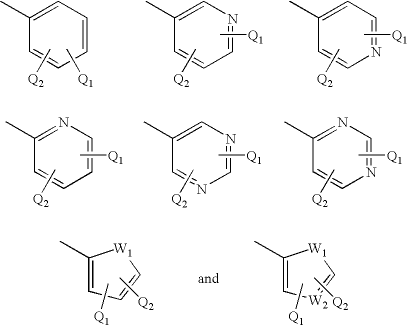

wherein A and B cannot simultaneously be a benzene ring;

Z is NO₂, CN, COOH, COR, NHCOR or CONHR;

Y is CF₃, F, I, Br, Cl, CN CR₃ or SnR₃;

Q₁ and Q₂ are independently of each other a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, SCN, NCS, OCN, NCO,

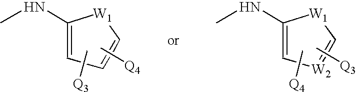

Q₃ and Q₄ are independently of each other a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R or SR, SCN, NCS, OCN, NCO;

W₁ is O, NH, NR, NO or S; and

W₂ is N or NO.

In another embodiment, the SARM is a compound represented by the structure of formula IV and/or an analog, derivative, metabolite, isomer, pharmaceutically acceptable salt, pharmaceutical product, hydrate, N-oxide, impurity, prodrug, polymorph, crystal, or any combination thereof

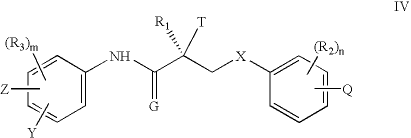

wherein X is a bond, O, CH₂, NH, Se, PR, NO or NR;

G is O or S;

T is OH, OR, —NHCOCH₃, or NHCOR;

R is alkyl, haloalkyl, dihaloalkyl, trihaloalkyl, CH₂F, CHF₂, CF₃, CF₂CF₃, aryl, phenyl, halogen, alkenyl or OH;

R₁ is CH₃, CH₂F, CHF₂, CF₃, CH₂CH₃, or CF₂CF₃;

R₂ is F, Cl, Br, I, CH₃, CF₃, OH, CN, NO₂, NHCOCH₃, NHCOCF₃, NHCOR, alkyl, arylalkyl, OR, NH₂, NHR, NR₂, SR, SCN, NCS, OCN, NCO;

R₃ is F, Cl, Br, I, CN, NO₂, COR, COOH, CONHR, CF₃, SnR₃, or R₃ together with the benzene ring to which it is attached forms a fused ring system represented by the structure:

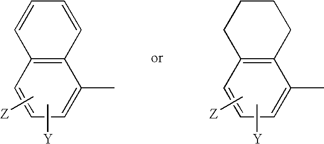

Z is NO₂, CN, COR, COOH, or CONHR;

Y is CF₃, F, Br, Cl, I, CN, or SnR₃;

Q is H, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OH, OR, COR, OCOR, OSO₂R, SO₂R, SR; or Q together with the benzene ring to which it is attached is a fused ring system represented by structure A, B or C:

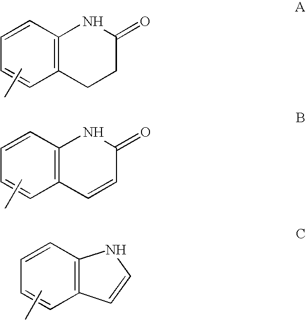

n is an integer of 1-4; and

m is an integer of 1-3.

In another embodiment, the SARM is a compound represented by the structure of formula V and/or an analog, derivative, metabolite, isomer, pharmaceutically acceptable salt, pharmaceutical product, hydrate, N-oxide, impurity, prodrug, polymorph, crystal, or any combination thereof

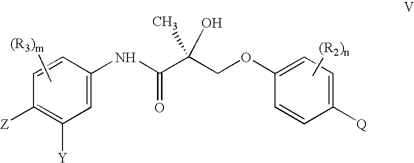

wherein

R₂ is F, Cl, Br, I, CH₃, CF₃, OH, CN, NO₂, NHCOCH₃, NHCOCF₃, NHCOR, alkyl, arylalkyl, OR, NH₂, NHR, NR₂, SR;

R₃ is F, Cl, Br, I, CN, NO₂, COR, COOH, CONHR, CF₃, SnR₃, or R₃ together with the benzene ring to which it is attached forms a fused ring system represented by the structure:

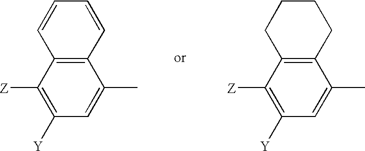

R is alkyl, haloalkyl, dihaloalkyl, trihaloalkyl, CH₂F, CHF₂, CF₃, CF₂CF₃, aryl, phenyl, halogen, alkenyl or OH;

Z is NO₂, CN, COR, COOH, or CONHR;

Y is CF₃ F, Br, Cl, I, CN, or SnR₃;

Q is H, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OH, OR, COR, OCOR, OSO₂R, SO₂R, SR; or Q together with the benzene ring to which it is attached is a fused ring system represented by structure A, B or C:

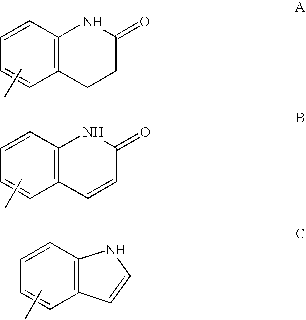

n is an integer of 1-4; and

m is an integer of 1-3.

In another embodiment, the SARM is a compound represented by the structure of formula VI and/or an analog, derivative, metabolite, isomer, pharmaceutically acceptable salt, pharmaceutical product, hydrate, N-oxide, impurity, prodrug, polymorph, crystal, or any combination thereof.

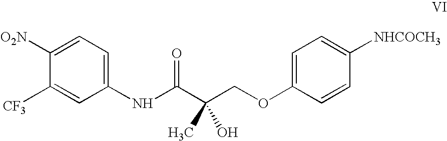

In another embodiment, the SARM is a compound represented by the structure of formula VII and/or an analog, derivative, metabolite, isomer, pharmaceutically acceptable salt, pharmaceutical product, hydrate, N-oxide, impurity, prodrug, polymorph, crystal, or any combination thereof.

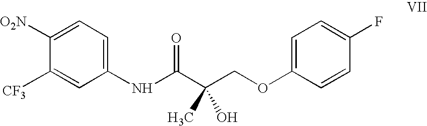

The novel selective androgen receptor modulator compounds of the present invention, either alone or as a pharmaceutical composition, are useful for a) male contraception; b) treatment of a variety of hormone-related conditions, for example conditions associated with ADAM, such as fatigue, depression, decreased libido, sexual dysfunction, erectile dysfunction, hypogonadism, osteoporosis, hair loss, obesity, sarcopenia, osteopenia, benign prostate hyperplasia, and alterations in mood and cognition; c) treatment of conditions associated with ADIF, such as sexual dysfunction, decreased sexual libido, hypogonadism, sarcopenia, osteopenia, osteoporosis, alterations in cognition and mood, depression, anemia, hair loss, obesity, endometriosis, breast cancer, uterine cancer and ovarian cancer; d) treatment and/or prevention of acute and/or chronic muscular wasting conditions; e) preventing and/or treating dry eye conditions; f) oral androgen replacement therapy; and/or g) decreasing the incidence of, halting or causing a regression of prostate cancer.

The selective androgen receptor modulator compounds of the present invention offer a significant advance over steroidal androgen treatment because the selective androgen receptor modulator compounds of the present invention have been shown in-vivo to have an androgenic and anabolic activity of a nonsteroidal ligand for the androgen receptor. Thus, the selective androgen receptor modulator compounds have an androgenic and anabolic activity of a nonsteroidal ligand for the androgen receptor and will not be accompanied by serious side effects, inconvenient modes of administration, or high costs and still have the advantages of oral bioavailability, lack of cross-reactivity with other steroid receptors, and long biological half-lives.

The present invention thus provides rapid, specific and sensitive assays for detecting, identifying, characterizing, purifying, obtaining structural information on, qualifying, screening, separating, fractionating and/or quantifying of SARMs, which methods are highly reliable, sensitive, specific and reproducible, and which is of great commercial value. The methods of the present invention allow better clinical evaluation and practice for a patient, and can be used used to monitor and reduce incidence of off-label use by athletes and others. Further, the methods of the present invention offer important tools to investigate forensic and overdose situations, evalulate black market and bootleg anabolic compounds that are produced illegally, and can further be used for disqualification of performers from competition when appropriate.

### BRIEF DESCRIPTION OF THE DRAWINGS

The present invention will be understood and appreciated more fully from the following detailed description taken in conjunction with the appended drawings in which:

FIG. 1: Concentrations of Compound VII in rat plasma after intravenous and oral administration of the drug at doses ranging from 0.1 to 30 mg/kg.

### DETAILED DESCRIPTION OF THE INVENTION

The present invention relates to analytical methods for detecting, identifying characterizing, purifying, obtaining structural information on, qualifying, screening, separating, fractionating and/or quantifying a Selective Androgen Receptor Modulator (SARM) compound in a sample. The methods of the present invention comprise obtaining a sample, and detecting the presence and/or concentration of a SARM compound in the sample. As demonstrated herein, the methods of the present invention are highly robust, sensitive, specific, reliable, validated, validatable and reproducible assays that are of great commercial potential and value.

In one embodiment, the sample is a biological sample. In another embodiment, the sample is a blood serum sample, a plasma sample, a urine sample, a CSF sample, a saliva sample, a fecal sample, an isolated or precipitated fraction, a protein adduct, or a protein extract.

The methods of the present invention comprise detecting the presence and/or concentration of a SARM compound in the sample. In one embodiment, the SARM compound is detected by subjecting an aliquot from the sample to any one or more of the following analytical detection methods: mass spectroscopy (MS), MS-MS, UV, IR, NMR, fluorescence, radiochemical detection, electrochemical detection, chemiluminscent detection, evaporative light scatter detection (ESLD), hyphenated techniques or methods, or any combination thereof. In one particular embodiment, the SARM compound is detected by measuring the UV absorbance of the SARM compound in the sample. In another particular embodiment, the SARM compound is detected by measuring the molecular ion peak of the SARM compound by mass spectrometry, for example by negative ion mass spectrometry.

In one embodiment, the method of the present invention further comprises subjecting the sample to a chromatographic separation prior to said detection step. In one embodiment, the chromatographic separation is by liquid chromatography (LC). In another embodiment, the chromatographic separation is by High Performance Liquid Chromatography (HPLC). In another embodiment, the chromatographic separation is by Thin Layer Chromatography (TLC). In another embodiment, the chromatographic separation is by capillary electrophoresis (CE). In another embodiment, the chromatographic separation is by microLC electrophoresis. In another embodiment, the chromatographic separation is by nano LC electrophoresis. In another embodiment, the chromatographic separation is by gel electrophoresis (GE). In another embodiment, the chromatographic separation is by isoelectric focusing gel electrophoresis. In another embodiment, the chromatographic separation is by sample concentration.

In one embodiment, the method of the present invention comprises separating the sample by chromatographic separation using any one of the detection methods described hereinabove, followed by a detection step using any one of the separation techniques described hereinabove. For example, in one embodiment, the method of the present invention comprises subjecting the sample to liquid chromatography (LC); and detecting the presence and/or concentration of a SARM compound in the sample by mass spectrometry (MS). In accordance with this embodiment, the method is a Liquid Chromatography/Mass Spectrometry method (LC/MS). In another embodiment, the mass spectrometry is negative ion mass spectrometry.

In another embodiment, the method of the present invention comprises subjecting the sample to High Performance Liquid Chromatography (HPLC); and detecting the presence and/or concentration of a SARM compound in the sample by measuring the UV absorbance of the SARM compound. In accordance with this embodiment, the method is a High Performance Liquid Chromatography/UV method (HPLC/UV).

In another embodiment, the method of the present invention comprises subjecting the sample to liquid chromatography (LC); and detecting the presence and/or concentration of a SARM compound in the sample by MS-MS. In accordance with this embodiment, the method is a LC/MS-MS method.

In another embodiment, the method of the present invention comprises subjecting the sample to capillary electrophoresis (CE); and detecting the presence and/or concentration of a SARM compound in the sample by mass spectrometry (MS). In accordance with this embodiment, the method is a CE/MS method.

It is apparent to a person skilled in the art that any other combination of separation/detection techniques is applicable, and is within the broad scope of the present Invention.

In one embodiment, the chromatography step comprises producing a chromatograph of the sample, the chromatograph comprising a series of peaks representing individual chemical compounds contained in the sample. The individual chemical compounds are automatically collected into separate tube, and the content of each tube is analyzed by any of the detection methods outlined above, for example by measuring the UV absorbance of an aliquot drawn from each tube, and/or by subjecting the different aliquots to mass spectrometric analysis. In another embodiment, the method further comprises the step of comparing output from the detection analysis for each tube with data identifying known SARM compounds expected to be present in the sample. In a particular embodiment, the data is stored in a database of a digital signal processor.

Liquid Chromatography/Mass Spectrometry (LC/MS)

In one embodiment, the present invention relates to an analytical chromatographic-mass spectrometric method of detecting, identifying characterizing, purifying, obtaining structural information on, qualifying, screening, separating, fractionating and/or quantifying a SARM compound in a sample, which comprises subjecting a sample, such as a biological sample, to chromatography, and detecting the presence and/or concentration of the SARM compound in the sample by mass spectrometry. In another embodiment, the mass spectrometry is negative ion mass spectrometry.

A mass spectrometer (MS) is a highly sensitive analysis instrument which provides the user with information on the molecular weight and structure of organic compounds and which is therefore indispensable in the fields of organic chemistry, pharmacy and biochemistry (see for example Llewellyn et al. U.S. Pat. No. 3,429,105, Haruki et al. U.S. Pat. No. 3,581,465, Saunders U.S. Pat. No. 3,662,520 and Grunnee et al. U.S. Pat. No. 3,678,656, among others).

However, MS cannot separate and distinguish the components of a mixture, and therefore when it is a mixture that is to be analyzed, analysis has been difficult. In view of this point, a chromatograph directly coupled mass spectrometer has been proposed.

When the chromatograph is a liquid chromatograph (LC), the method is a Liquid Chromatography/Mass Spectrometry (LC/MS) method. In one embodiment, the chromatography is liquid chromatography, and the method is a Liquid Chromatography/Mass Spectrometry method (LC/MS). A liquid chromatograph (LC) is superior in separating and distinguishing a mixture, and takes advantage of the fact that non-volatile substances, thermally unstable substances, inorganic and organic compounds, low and high molecular weight substances can be analyzed easily if only they are soluble in solvents. Liquid chromatography techniques for analyzing complex mixtures are known in the art, (see for example to Bakalyar et al. U.S. Pat. No. 3,446,057, Skeggs U.S. Pat. No. 3,230,048, and Waters U.S. Pat. Nos. 3,522,725 and 3,537,585).

LC is a device for mixing a sample for analysis with a solvent and separating the mixture under atmospheric pressure, while MS is a device for analyzing an ionized sample in high vacuum. For coupling the two, therefore, it is necessary to remove the solvent (desolvation) from LC effluent, then ionizing the sample remaining after desolvation and feeding the ionized sample to MS held in high vacuum. A technique for coupling LC and MS is described, for example, in Japanese Patent Publication No.43692/83, in which effluent from LC is nebulized, the resulting mists are desolvated and ionized, and the thus-ionized sample (desolvated effluent) is subjected to mass spectrometric analysis.

It has also been proposed to analyze complex mixtures in a strictly batch-wise manner. A certain volume of the liquid chromatography effluent is collected, the solvent is evaporated off, and the residue is introduced into the mass spectrometer. R. E. Lovins, S. R. Ellis, G. D. Talbert and C. R. McKinney, Anal. Chem., 45, 1553 (1973).

Accordingly, in one embodiment, the method of the present invention comprises subjecting a sample, such as a biological sample suspected of containing a SARM compound to liquid chromatography; and detecting the presence and/or concentration of the SARM compound in the sample by mass spectrometry. In on embodiment, the chromatography step comprises producing a chromatograph of the sample, the chromatograph comprising a series of peaks representing individual chemical compounds contained in the sample. The individual chemical compounds are automatically collected into separate tube, and the content of each tube is analyzed by mass spectrometry. In another embodiment, the method further comprises the step of comparing output from the mass spectrometric analysis for each tube with data identifying known SARM compounds expected to be present in the sample. In a particular embodiment, the data is stored in a database of a digital signal processor.

High Performace Liquid Chromatrography (HPLC)

In another embodiment, the present invention relates to an analytical method of detecting, identifying characterizing, purifying, obtaining structural information on, qualifying, screening, separating, fractionating and/or quantifying a SARM compound in a sample, which comprises subjecting a sample, such as a biological sample, to High Performance Liquid Chromatography (HPLC), and detecting the presence and/or concentration of the SARM compound in the sample, using any detection method discussed above. In one embodiment, the SARM is detected by measuring the UV absorbance of the compound. In accordance with this embodiment, the method is a High Performance Liquid Chromatography/UV (HPLC/UV).

High performance liquid chromatography (HPLC) has become one of the most useful analytical tools in a modern chemical laboratory. It is particularly useful for the room temperature separation and analysis of very small amounts of organic or biochemical compounds. Most commonly, in HPLC a spectrophotometer is used to detect the presence of the analyte, since most organic compounds will absorb radiation in the range of about 190 nm to 800 nm, particularly in the UV range of about 190 nm to 350 nm.

HPLC is performed in a pressure-resistant tube containing a stationary adsorbent which is the packing material. A pressure mechanism exerts pressure on a mobile phase applied to one end of the column and moves it through the column causing it to exit the opposite end of the column. A sample containing a mixture of compounds is injected onto the column through a sample injection port. As the sample moves through the packing material, the various components of the sample adsorb to the packing material with different affinities. The components, therefore, can elute from the column separately under appropriate conditions. On a reverse phase HPLC column the compounds within a sample are separated based on hydrophobicity.

HPLC analysis may be performed in isocratic or gradient mode. An isocratic HPLC separation is one which is carried out under a constant eluant composition. A gradient HPLC separation is characterized by a gradual change in the percentage of two or more solvents applied to the column over time. The change in solvent often is controlled by a mixing device which mixes solvent A and solvent B to produce the HPLC solvent just prior to its movement through the column. The amount of time over which the gradient is changed from one extreme to the opposite extreme is the gradient time.

HPLC columns can be obtained from a variety of commercial sources, such as Phenomenex, Waters, and Merck.

The HPLC column is packed with a non-polar stationary phase on solid support particles. Prior to loading the sample on the column the column is conditioned by flowing through it the intended mobile phase, which can be an isocratic mobile phase or a gradient mobile phase. Once the column is conditioned, the system is initiated by the injection of the sample. As the small organic compounds contact the non-polar packing material, each molecule is adsorbed to the packing material. The affinity with which each compound adsorbs to the packing material is dependent on the hydrophobicity of the individual compound. The injection of the sample defines time zero for the run.

An isocratic mobile phase or a gradient mobile phase is applied to the column immediately after the injection of the sample in order to elute the compounds bound therein in distinct fractions. A gradient HPLC system includes two reservoirs, each containing a different polarity solvent, which are pumped through a mixing chamber over the column by means of a pump. A gradient mobile phase is then applied to the column, by increasing the polarity of the gradient over a period of time. Based on the polarity of the stationary phase, the mobile phase and the compounds in the mixture, the compounds are eluted from the stationary phase. Solvents typically used for gradients in reversed phase HPLC generally include acetonitrile, methanol, isopropanol and propanol. Modifiers are typically added to the mobile phase, primarily to buffer the pH to a certain narrow range, and include a variety of acids and bases such as phosphoric acid, perfluorinated carboxylic acids and amines.

An HPLC compatible detector is used to detect the presence of small organic compounds as they are eluted from the column. A compatible detector is one which is capable of detecting a signal from a compound in an eluant and which produces a signal to indicate the presence of that compound. HPLC compatible detectors include, but are not limited to, fluorescent, electrochemical, IR, NMR, chemiluminescent, UV and mass spectrometry.

In another embodiment, the method of the present invention further comprises the step of quantifying the amount of SARM compound in the sample by comparing the amount obtained from the sample with a reference sample comprising known amounts of a reference SARM compound. As a reference SARM compound, any one or more of the SARM compounds described below are applicable.

Methods of determining concentrations of compounds are well known to a person skilled in the art. For example, the amount of the compound in the sample is determined, and the concentration of the compound in the sample is determined by comparing the level with a standard sample containing a known concentration of the SARM compound or any reference SARM compound. Calibration curves of known concentrations of the compound in the sample, can be obtained, and the concentration of the compound in the test sample is calculated therefrom.

In another embodiment, the compound is detected in the sample by contacting the sample with a binding protein which specifically binds to the compound. The concentration determining the amount of binding protein bound to the compound. The concentration of the compound can be determined by measuring the amount of binding protein bound to the compound, and comparing that amount to a standard sample containing a known concentration of the SARM compound or reference SARM compound—binding protein complex.

Protein levels can be determined according to standard techniques, as described in Sambrook et al. Briefly, a sample obtained from a subject is contacted with a binding protein which specifically binds to a specific compound of the present invention, and the amount of complex formed between the binding protein and the compound is determined. In one embodiment, the binding protein is an antibody which specifically binds to one or more compounds of the present invention. In another embodiment, the binding protein has a detectable label bound thereto, and the complex between the binding protein-label compound is determined by visualizing the complex.

As defined herein, “contacting” means that the binding protein is introduced into the sample in a test tube, flask, tissue culture, chip, array, plate, microplate, capillary, or the like, and incubated at a temperature and time sufficient to permit the binding component to bind to a cell or a fraction thereof or plasma/serum or a fraction thereof containing the target. Methods for contacting the samples with the binding proteins, or other specific binding components are known to those skilled in the art and may be selected depending on the type of assay protocol to be run. Incubation methods are also standard and are known to those skilled in the art.

“Visualizing” the complex may be carried out by any means known in the art, including, but not limited to, ELISA, radioimmunoassay, flow cytometry, dot blots, western immunoblotting combined with gel electrophoresis (GE), immunohistochemistry at light and electron pe levels, HPLC and mass spectrometry.

Either monoclonal or polyclonal antibodies (as well as any recombinant antibodies) specific for the selective androgen modulator compounds or the non-steroidal agonist compounds of the present invention can be used in the various immunoassays. The antibodies may be detectably labeled, utilizing conventional labeling techniques well-known to the art. As used herein, the term “label” refers to a molecule, which may be conjugated or otherwise attached (i.e., covalently or non-covalently) to a binding protein as defined herein. Labels are known to those skilled in the art. Thus, the antibodies may be labeled with radioactive isotopes, non-radioactive isotopic labels, fluorescent labels, enzyme labels, chemiluminescent labels, bioluminescent labels, free radical labels, or bacteriophage labels, using techniques known in the art. Examples of radioisotopic labels are ³H, ¹²⁵I, ¹³¹I, ³⁵S, ¹⁴C, etc. Examples of non-radioactive isotopic labels are ⁵⁵Mn, ⁵⁶Fe, etc. Examples of fluorescence labels are fluorescent labels which are directly labeled with the preferred fluorescence label, or fluorescent labels which are indirectly labeled with the preferred fluorescence label. In the last case, the preferred fluorescence label is conjugated to a secondary antibody, which is directed against the first antibody, such as an anti species Ig antibody. Typical fluorescent labels include, but are not limited to a fluorescein label, an isothiocyanate label, a rhodamine label, a phycoerythrin label, etc., for example fluorescein isothiocyanate (FITC, International Biological Supplies, Melbourne, Fla.), rhodamine, phycoerythrin (P.E., Coulter Corp., Hialeah, Fla.), phycocyanin, alophycocyanin, phycoerythrin-cyanin dye 5 (PECy5, Coulter), label, a phycocyanin label, an allophycocyanin label, an O-phthaldehyde label, a fluorescamine and Texas Red.

Examples of enzyme labels include alkaline phosphatase, beta-galactosidase, glucose-6-phosphate dehydrogenase, maleate dehydrogenase, and peroxidase. Two principal types of enzyme immunoassay are the enzyme-linked immunosorbent assay (ELISA), an d the homogeneous enzyme immunoassay, also known as enzyme-multiplied immunoassay (EMIT, Syva Corporation, Palo Alto, Calif.). In the ELISA system, separation may be achieved, for example, by the use of antibodies coupled to a solid phase. The EMIT system depends on deactivation of the enzyme in the tracer-antibody complex; the activity can thus be measured without the need for a separation step.

Particularly suitable labels include those, which permit analysis by flow cytometry, e.g., fluorochromes. Other suitable detectable labels include those useful in colorimetric enzyme systems, e. g., horseradish peroxidase (HRP) and alkaline phosphatase (AP). Other proximal enzyme systems are known to those of skill in the art, including hexokinase in conjunction with glucose-6-phosphate dehydrogenase.

Additionally, chemiluminescent compounds may be used as labels. Chemiluminescent labels, such as green fluorescent proteins, blue fluorescent proteins, and variants thereof are known. Also bioluminescence or chemiluminescence can be detected using, respectively, NAD oxidoreductase with luciferase and substrates NADH and FNIN or peroxidase with luminol and substrate peroxide. Typical chemiluminescent compounds include luminol, isoluminol, aromatic acridinium esters, imidazoles, acridinium salts, and oxalate esters. Similarly, bioluminescent compounds may be utilized for labelling, the bioluminescent compounds including luciferin, luciferase, and aequorin. Once labeled, the antibody may be employed to identify and quantify immunologic counterparts (antibody or antigenic polypeptide) utilizing techniques well-known to the art.

Selective Androgen Receptor Modulators

Selective androgen receptor modulator (SARM) compounds are a new class of nonsteroidal compounds which have been shown to be useful for a) male contraception; b) treatment of a variety of hormone-related conditions, for example conditions associated with Androgen Decline in Aging Male (ADAM), such as fatigue, depression, decreased libido, sexual dysfunction, erectile dysfunction, hypogonadism, osteoporosis, hair loss, anemia, obesity, sarcopenia, osteopenia, osteoporosis, benign prostate hyperplasia, alterations in mood and cognition and prostate cancer; c) treatment of conditions associated with ADIF, such as sexual dysfunction, decreased sexual libido, hypogonadism, sarcopenia, osteopenia, osteoporosis, alterations in cognition and mood, depression, anemia, hair loss, obesity, endometriosis, breast cancer, uterine cancer and ovarian cancer; d) treatment and/or prevention of acute and/or chronic muscular wasting conditions; e) preventing and/or treating dry eye conditions; f) oral androgen replacement therapy; and/or g) decreasing the incidence of, halting or causing a regression of prostate cancer.

Several appropriately substituted SARM compounds have been shown to have an unexpected selective in-vivo androgenic and anabolic activity of a nonsteroidal ligand for the androgen receptor (AR).

Steroid hormones are one example of small hydrophobic molecules (ligands) that diffuse directly across the plasma membrane of target cells and bind to intracellular cell signaling receptors. These receptors are structurally related and constitute the intracellular receptor superfamily (or steroid-hormone receptor superfamily). Steroid hormone receptors include progesterone receptors, estrogen receptors, androgen receptors, glueocorticoid receptors, and mineralocorticoid receptors. As used herein, the term “receptor” also encompasses recombinant receptors. The compounds described herein are androgen receptor ligands.

In addition to ligand binding to the receptors, the receptors can be blocked to prevent ligand binding. When a substance binds to a receptor, the three-dimensional structure of the substance fits into a space created by the three-dimensional structure of the receptor in a ball and socket configuration. The better the ball fits into the socket, the more tightly it is held. This phenomenon is called affinity. If the affinity of a substance is greater than the original hormone, it will compete with the hormone and bind the binding site more frequently. Once bound, signals may be sent through the receptor into the cells, causing the cell to respond in some fashion. This is called activation. On activation, the activated receptor then directly regulates the transcription of specific genes. But the substance and the receptor may have certain attributes, other than affinity, in order to activate the cell. Chemical bonds between atoms of the substance and the atoms of the receptors may form. In some cases, this leads to a change in the configuration of the receptor, which is enough to begin the activation process (called signal transduction).

In one embodiment, the appropriately substituted selective androgen receptor modulator compounds described herein are agonist compounds. A receptor agonist is a substance, which binds receptors and activates them. Thus, in one embodiment, the SARM compounds described herein are useful in binding to and activating steroidal hormone receptors. Thus, in one embodiment, the SARM compounds described herein are useful in binding to and activating the androgen receptor. In another embodiment, the agonist compound also has anabolic activity. In another embodiment, the compounds described herein are selective androgen modulator compounds, which have agonistic, and anabolic activity of a nonsteroidal compound for the androgen receptor.

In another embodiment, other appropriately substituted selective androgen receptor modulator compounds described herein are antagonist compounds. A receptor antagonist is a substance, which binds receptors and inactivates them. Thus, in one embodiment, the SARM compounds described herein are useful in binding to and inactivating steroidal hormone receptors. In one embodiment, the antagonist compound is an antagonist, which binds the androgen receptor.

In yet another embodiment, the SARM compounds described herein can be classified as partial AR agonist/antagonists. The SARMs are AR agonists in some tissues, to cause increased transcription of AR-responsive genes (e.g. muscle anabolic effect). In other tissues, these compounds serve as inhibitors at the AR to prevent agonistic effects of the native androgens.

Assays to determine whether SARM compounds are AR agonists or antagonists are well known to a person skilled in the art. For example, AR agonistic activity can be determined by monitoring the ability of the SARM compounds to maintain and/or stimulate the growth of AR containing tissue such as prostate and seminal vesicles, as measured by weight. AR antagonistic activity can be determined by monitoring the ability of the SARM compounds to inhibit the growth of AR containing tissue.

The SARM compounds described herein bind either reversibly or irreversibly to an androgen receptor. In one embodiment, the androgen receptor is an androgen receptor of a mammal. In another embodiment, the androgen receptor is an androgen receptor of a human. In one embodiment, the SARM compounds bind reversibly to the androgen receptor of a mammal, for example a human. Reversible binding of a compound to a receptor means that a compound can detach from the receptor after binding.

In another embodiment, the SARM compounds bind irreversibly to the androgen receptor of a mammal, for example a human. Thus, in one embodiment, the SARM compounds may contain a functional group (e.g. affinity label) that allows alkylation of the androgen receptor (i.e. covalent bond formation). Thus, in this case, the compounds are alkylating agents which bind irreversibly to the receptor and, accordingly, cannot be displaced by a steroid, such as the endogenous ligands DHT and testosterone. An “alkylating agent” is defined herein as an agent which alkylates (forms a covalent bond) with a cellular component, such as DNA, RNA or enzyme. It is a highly reactive chemical that introduces alkyl radicals into biologically active molecules and thereby prevents their proper functioning. The alkylating moiety is an electrophilic group that interacts with nucleophilic moieties in cellular components.

As contemplated herein, specific non-limiting examples of SARM compounds are:

In one embodiment, the SARM is a compound represented by the structure of formula I:

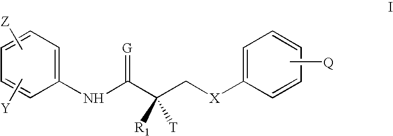

wherein G is O or S;

X is a bond, O, CH₂, NH, Se, PR, NO or NR;

T is OH, OR, —NHCOCH₃, or NHCOR

Z is NO₂, CN, COOH, COR, NHCOR or CONHR;

Y is CF₃, F, I, Br, Cl, CN, CR₃ or SnR₃;

Q is alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, SCN, NCS, OCN, NCO; or Q together with the benzene ring to which it is attached is a fused ring system represented by structure A, B or C:

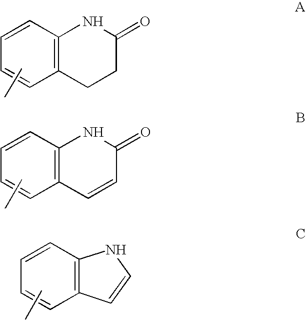

R is alkyl, haloalkyl, dihaloalkyl, trihaloalkyl, CH₂F, CHF₂, CF₃, CF₂CF₃, aryl, phenyl, halogen, alkenyl or OH; and

R₁ is CH₃, CH₂F, CHF₂, CF₃, CH₂CH₃, or CF₂CF₃.

In one embodiment, the SARM is an analog of the compound of formula I. In another embodiment, the SARM is a derivative of the compound of formula I. In another embodiment, the SARM is an isomer of the compound of formula I. In another embodiment, the SARM is a metabolite of the compound of formula I. In another embodiment, the SARM is a pharmaceutically acceptable salt of the compound of formula I. In another embodiment, the SARM is a pharmaceutical product of the compound of formula I. In another embodiment, the SARM is a hydrate of the compound of formula I. In another embodiment, the SARM is an N-oxide of the compound of formula I. In another embodiment, the SARM is a prodrug of the compound of formula I. In another embodiment, the SARM is a crystal of the compound of formula I. In another embodiment, the SARM is a polymorph of the compound of formula I. In another embodiment, the SARM is an impurity of the compound of formula I. In another embodiment, the SARM is a combination of any of an analog, derivative, metabolite, isomer, pharmaceutically acceptable salt, pharmaceutical product, hydrate N-oxide, impurity, prodrug, polymorph or crystal of the compound of formula I.

In one embodiment, the SARM compound is a compound of formula I wherein X is O. In one embodiment, the SARM compound is a compound of formula I wherein G is O. In another embodiment, the SARM compound is a compound of formula I wherein Z is NO₂. In another embodiment, the SARM compound is a compound of formula I wherein Z is CN. In another embodiment, the SARM compound is a compound of formula I wherein Y is CF₃. In another embodiment, the SARM compound is a compound of formula I wherein Q is NHCOCH₃. In another embodiment, the SARM compound is a compound of formula I wherein Q is F. In another embodiment, the SARM compound is a compound of formula I wherein T is OH. In another embodiment, the SARM compound is a compound of formula I wherein R₁ is CH₃.

In another embodiment, the SARM is a compound represented by the structure of formula II:

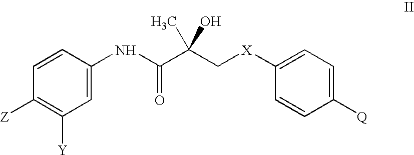

wherein X is a bond, O, CH₂, NH, Se, PR, NO or NR;

Z is NO₂, CN, COOH, COR, NHCOR or CONHR;

Y is CF₃, F, I, Br, Cl, CN, CR₃ or SnR₃;

Q is alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, SCN, NCS, OCN, NCO; or Q together with the benzene ring to which it is attached is a fused ring system represented by structure A, B or C:

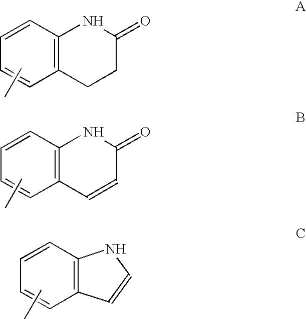

R is alkyl, haloalkyl, dihaloalkyl, trihaloalkyl, CH₂F, CHF₂, CF₃, CF₂CF₃, aryl, phenyl, halogen, alkenyl or OH.

In one embodiment, the SARM is an analog of the compound of formula II. In another embodiment, the SARM is a derivative of the compound of formula II. In another embodiment, the SARM is an isomer of the compound of formula II. In another embodiment, the SARM is a metabolite of the compound of formula II. In another embodiment, the SARM is a pharmaceutically acceptable salt of the compound of formula II. In another embodiment, the SARM is a pharmaceutical product of the compound of formula II. In another embodiment, the SARM is a hydrate of the compound of formula II. In another embodiment, the SARM is an N-oxide of the compound of formula II. In another embodiment, the SARM is a prodrug of the compound of formula II. In another embodiment, the SARM is a crystal of the compound of formula II. In another embodiment, the SARM is a polymorph of the compound of formula II. In another embodiment, the SARM is an impurity of the compound of formula II. In another embodiment, the SARM is a combination of any of an analog, derivative, metabolite, isomer, pharmaceutically acceptable salt, pharmaceutical product, hydrate N-oxide, impurity, prodrug, polymorph or crystal of the compound of formula II.

In one embodiment, the SARM compound is a compound of formula II wherein X is O. In another embodiment, the SARM compound is a compound of formula II wherein Z is NO₂. In another embodiment, the SARM compound is a compound of formula II wherein Z is CN. In another embodiment, the SARM compound is a compound of formula II wherein Y is CF₃. In another embodiment, the SARM compound is a compound of formula II wherein Q is NHCOCH₃. In another embodiment, the SARM compound is a compound of formula II wherein Q is F.

In another embodiment, the SARM is a compound represented by the structure of formula III:

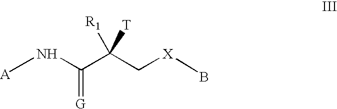

wherein X is a bond, O, CH₂, NH, Se, PR, NO or NR;

G is O or S;

R₁ is CH₃, CH₂F, CHF₂, CF₃, CH₂CH₃, or CF₂CF₃;

T is OH, OR, —NHCOCH₃, or NHCOR;

R is alkyl, haloalkyl, dihaloalkyl, trihaloalkyl, CH₂F, CHF₂, CF₃, CF₂CF₃, aryl, phenyl, halogen, alkenyl or OH;

A is a ring selected from:

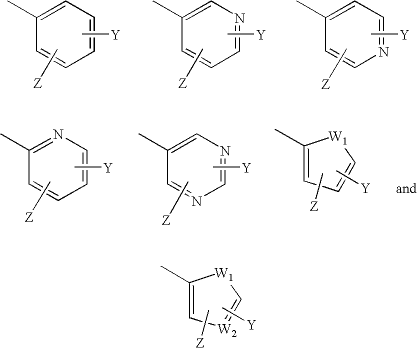

B is a ring selected from:

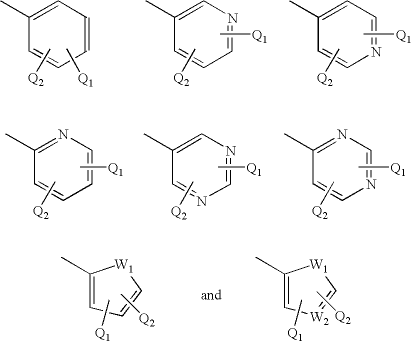

wherein A and B cannot simultaneously be a benzene ring;

Z is NO₂, CN, COOH, COR, NHCOR or CONHR;

Y is CF₃, F, I, Br, Cl, CN CR₃ or SnR₃;

Q₁ and Q₂ are independently of each other a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, SCN, NCS, OCN, NCO,

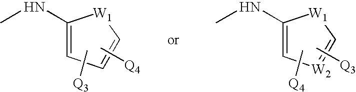

Q₃ and Q₄ are independently of each other a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R or SR, SCN, NCS, OCN, NCO;

W₁ is O, NH, NR, NO or S; and

W₂ is N or NO.

In one embodiment, the SARM is an analog of the compound of formula III. In another embodiment, the SARM is a derivative of the compound of formula I. In another embodiment, the SARM is an isomer of the compound of formula I. In another embodiment, the SARM is a metabolite of the compound of formula III. In another embodiment, the SARM is a pharmaceutically acceptable salt of the compound of formula III. In another embodiment, the SARM is a pharmaceutical product of the compound of formula III. In another embodiment, the SARM is a hydrate of the compound of formula III. In another embodiment, the SARM is an N-oxide of the compound of formula III. In another embodiment, the SARM is a prodrug of the compound of formula III. In another embodiment, the SARM is a crystal of the compound of formula III. In another embodiment, the SARM is a polymorph of the compound of formula III. In another embodiment, the SARM is an impurity of the compound of formula III. In another embodiment, the SARM is a combination of any of an analog, derivative, metabolite, isomer, pharmaceutically acceptable salt, pharmaceutical product, hydrate N-oxide, impurity, prodrug, polymorph or crystal of the compound of formula III.

In one embodiment, the SARM compound is a compound of formula III wherein X is O. In another embodiment, the SARM compound is a compound of formula III wherein G is O. In another embodiment, the SARM compound is a compound of formula I wherein T is OH. In another embodiment, the SARM compound is a compound of formula III wherein R₁ is CH₃. In another embodiment, the SARM compound is a compound of formula III wherein Z is NO₂. In another embodiment, the SARM compound is a compound of formula III wherein Z is CN. In another embodiment, the SARM compound is a compound of formula III wherein Y is CF₃. In another embodiment, the SARM compound is a compound of formula III wherein Q₁ is NHCOCH₃. In another embodiment, the SARM compound is a compound of formula III wherein Q₁ is F.

The substituents Z and Y can be in any position of the ring carrying these substituents (hereinafter “A ring”). In one embodiment, the substituent Z is in the para position of the A ring. In another embodiment, the substituent Y is in the meta position of the A ring. In another embodiment, the substituent Z is in the para position of the A ring and substituent Y is in the meta position of the A ring.

The substituents Q₁ and Q₂ can be in any position of the ring carrying these substituents (hereinafter “B ring”). In one embodiment, the substituent Q₁ is in the para position of the B ring. In another embodiment, the substituent is Q₂ is H. In another embodiment, the substituent Q₁ is in the para position of the B ring and the substituent is Q₂ is H. In another embodiment, the substituent Q₁ is NHCOCH₃ and is in the para position of the B ring, and the substituent is Q₂ is H.

In another embodiment, the SARM is a compound represented by the structure of formula IV:

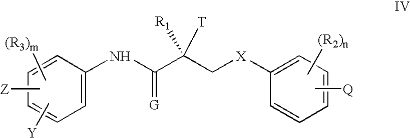

wherein X is a bond, O, CH₂, NH, Se, PR, NO or NR;

G is O or S;

T is OH, OR, —NHCOCH₃, or NHCOR;

R is alkyl, haloalkyl, dihaloalkyl, trihaloalkyl, CH₂F, CHF₂, CF₃, CF₂CF₃, aryl, phenyl, halogen, alkenyl or OH;

R₁ is CH₃, CH₂F, CHF₂, CF₃, CH₂CH₃, or CF₂CF₃;

R₂ is F, Cl, Br, I, CH₃, CF₃, OH, CN, NO₂, NHCOCH₃, NHCOCF₃, NHCOR, alkyl, arylalkyl, OR, NH₂, NHR, NR₂, SR, SCN, NCS, OCN, NCO;

R₃ is F, Cl, Br, I, CN, NO₂, COR, COOH, CONHR, CF₃, SnR₃, or R₃ together with the benzene ring to which it is attached forms a fused ring system represented by the structure:

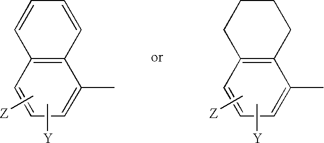

Z is NO₂, CN, COR, COOH, or CONHR;

Y is CF₃,F, Br, Cl, I, CN, or SnR₃;

Q is H, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OH, OR, COR, OCOR, OSO₂R, SO₂R, SR; or Q together with the benzene ring to which it is attached is a fused ring system represented by structure A, B or C:

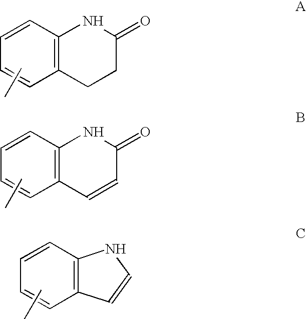

n is an integer of 1-4; and

m is an integer of 1-3.

In one embodiment, the SARM is an analog of the compound of formula IV. In another embodiment, the SARM is a derivative of the compound of formula IV. In another embodiment, the SARM is an isomer of the compound of formula IV. In another embodiment, the SARM is a metabolite of the compound of formula IV. In another embodiment, the SARM is a pharmaceutically acceptable salt of the compound of formula IV. In another embodiment, the SARM is a pharmaceutical product of the compound of formula IV. In another embodiment, the SARM is a hydrate of the compound of formula IV. In another embodiment, the SARM is an N-oxide of the compound of formula IV. In another embodiment, the SARM is a prodrug of the compound of formula IV. In another embodiment, the SARM is a crystal of the compound of formula IV. In another embodiment, the SARM is a polymorph of the compound of formula IV. In another embodiment, the SARM is an impurity of the compound of formula IV. In another embodiment, the SARM is a combination of any of an analog, derivative, metabolite, isomer, pharmaceutically acceptable salt, pharmaceutical product, hydrate N-oxide, impurity, prodrug, polymorph or crystal of the compound of formula IV.

In one embodiment, the SARM compound is a compound of formula IV wherein X is O. In another embodiment, the SARM compound is a compound of formula IV wherein G is O. In another embodiment, the SARM compound is a compound of formula IV wherein Z is NO₂. In another embodiment, the SARM compound is a compound of formula IV wherein Z is CN. In another embodiment, the SARM compound is a compound of formula IV wherein Y is CF₃. In another embodiment, the SARM compound is a compound of formula IV wherein Q is NHCOCH₃. In another embodiment, the SARM compound is a compound of formula IV wherein Q is F. In another embodiment, the SARM compound is a compound of formula IV wherein T is OH. In another embodiment, the SARM compound is a compound of formula IV wherein R₁ is CH₃. In another embodiment, the SARM compound is a compound of formula IV wherein Q is F and R₂ is CH₃. In another embodiment, the SARM compound is a compound of formula IV wherein Q is F and R₂ is Cl.

The substituents Z, Y and R₃ can be in any position of the ring carrying these substituents (hereinafter “A ring”). In one embodiment, the substituent Z is in the para position of the A ring. In another embodiment, the substituent Y is in the meta position of the A ring. In another embodiment, the substituent Z is in the para position of the A ring and substituent Y is in the meta position of the A ring.

The substituents Q and R₂ can be in any position of the ring carrying these substituents (hereinafter “B ring”). In one embodiment, the substituent Q is in the para position of the B ring. In another embodiment, the substituent Q is in the para position of the B ring. In another embodiment, the substituent Q is NHCOCH₃ and is in the para position of the B ring.

As contemplated herein, when the integers m and n are greater than one, the substituents R₂ and R₃ are not limited to one particular substituent, and can be any combination of the substituents listed above.

In another embodiment, the SARM is a compound represented by the structure of formula V:

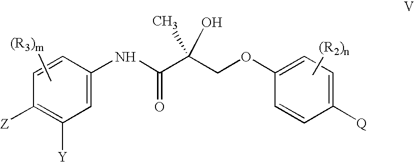

wherein

R₂ is F, Cl, Br, I, CH₃, CF₃, OH, CN, NO₂, NHCOCH₃, NHCOCF₃, NHCOR, alkyl, arylalkyl, OR, NH₂, NHR, NR₂, SR;

R₃ is F, Cl, Br, I, CN, NO₂, COR, COOH, CONHR, CF₃, SnR₃, or R₃ together with the benzene ring to which it is attached forms a fused ring system represented by the structure:

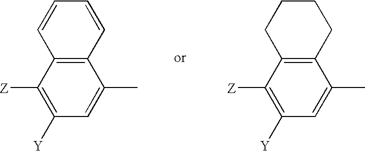

R is alkyl, haloalkyl, dihaloalkyl, trihaloalkyl, CH₂F, CHF₂, CF₃, CF₂CF₃, aryl, phenyl, halogen, alkenyl or OH;

Z is NO₂, CN, COR, COOH, or CONHR;

Y is CF₃,F, Br, Cl, I, CN, or SnR₃;

Q is H, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OH, OR, COR, OCOR, OSO₂R, SO₂R, SR; or Q together with the benzene ring to which it is attached is a fused ring system represented by structure A, B or C:

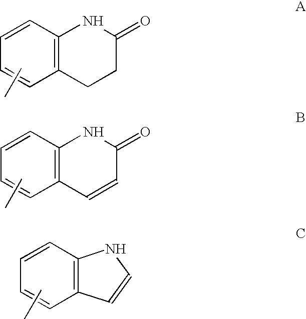

n is an integer of 1-4; and

m is an integer of 1-3.

In one embodiment, the SARM is an analog of the compound of formula V. In another embodiment, the SARM is a derivative of the compound of formula V. In another embodiment, the SARM is an isomer of the compound of formula V. In another embodiment, the SARM is a metabolite of the compound of formula V. In another embodiment, the SARM is a pharmaceutically acceptable salt of the compound of formula V. In another embodiment, the SARM is a pharmaceutical product of the compound of formula V. In another embodiment, the SARM is a hydrate of the compound of formula V. In another embodiment, the SARM is an N-oxide of the compound of formula V. In another embodiment, the SARM is a prodrug of the compound of formula V. In another embodiment, the SARM is a crystal of the compound of formula V. In another embodiment, the SARM is a polymorph of the compound of formula V. In another embodiment, the SARM is an impurity of the compound of formula V. In another embodiment, the SARM is a combination of any of an analog, derivative, metabolite, isomer, pharmaceutically acceptable salt, pharmaceutical product, hydrate N-oxide, impurity, prodrug, polymorph or crystal of the compound of formula V.

In another embodiment, the SARM is a compound of formula V wherein Z is NO₂. In another embodiment, the SARM is a compound of formula V wherein Z is CN. In another embodiment, the SARM is a compound of formula V wherein Y is CF₃. In another embodiment, the SARM is a compound of formula V wherein Q is NHCOCH₃. In another embodiment, the SARM is a compound of formula V wherein Q is F. In another embodiment, the SARM is a compound of formula V wherein Q is F and R₂ is CH₃. In another embodiment, the SARM is a compound of formula V wherein Q is F and R₂ is Cl.

The substituents Z, Y and R₃ can be in any position of the A ring, and he substituents Q and R₂ can be in any position of B ring, as discussed above for compound IV. Furthermore, as discussed above, when the integers m and n are greater than one, the substituents R₂ and R₃ are not limited to one particular substituent, and can be any combination of the substituents listed above.

In another embodiment, the SARM is a compound represented by the structure of formula VI.

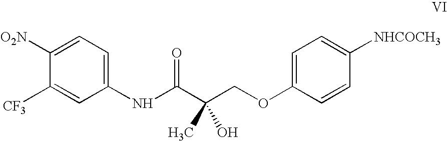

In one embodiment, the SARM is an analog of the compound of formula VI. In another embodiment, the SARM is a derivative of the compound of formula VI. In another embodiment, the SARM is an isomer of the compound of formula VI. In another embodiment, the SARM is a metabolite of the compound of formula VI. In another embodiment, the SARM is a pharmaceutically acceptable salt of the compound of formula VI. In another embodiment, the SARM is a pharmaceutical product of the compound of formula VI. In another embodiment, the SARM is a hydrate of the compound of formula VI. In another embodiment, the SARM is an N-oxide of the compound of formula VI. In another embodiment, the SARM is a prodrug of the compound of formula VI. In another embodiment, the SARM is a crystal of the compound of formula VI. In another embodiment, the SARM is a polymorph of the compound of formula VI. In another embodiment, the SARM is an impurity of the compound of formula VI. In another embodiment, the SARM is a combination of any of an analog, derivative, metabolite, isomer, pharmaceutically acceptable salt, pharmaceutical product, hydrate N-oxide, impurity, prodrug, polymorph or crystal of the compound of formula VI.

In another embodiment, the SARM is a compound represented by the structure of formula VII.

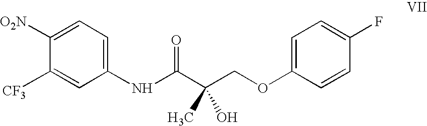

In one embodiment, the SARM is an analog of the compound of formula VII. In another embodiment, the SARM is a derivative of the compound of formula VII. In another embodiment, the SARM is an isomer of the compound of formula VII. In another embodiment, the SARM is a metabolite of the compound of formula VII. In another embodiment, the SARM is a pharmaceutically acceptable salt of the compound of formula VII. In another embodiment, the SARM is a pharmaceutical product of the compound of formula VII. In another embodiment, the SARM is a hydrate of the compound of formula VII. In another embodiment, the SARM is an N-oxide of the compound of formula VII. In another embodiment, the SARM is a prodrug of the compound of formula VII. In another embodiment, the SARM is a crystal of the compound of formula VII. In another embodiment, the SARM is a polymorph of the compound of formula VII. In another embodiment, the SARM is an impurity of the compound of formula VII. In another embodiment, the SARM is a combination of any of an analog, derivative, metabolite, isomer, pharmaceutically acceptable salt, pharmaceutical product, hydrate N-oxide, impurity, prodrug, polymorph or crystal of the compound of formula VII.

The substituent R is defined herein as an alkyl, haloalkyl, dihaloalkyl, trihaloalkyl, CH₂F, CHF₂, CF₃, CF₂CF₃; aryl, phenyl, halogen, alkenyl, or hydroxyl (OH).

An “alkyl” group refers to a saturated aliphatic hydrocarbon, including straight-chain, branched-chain and cyclic alkyl groups. In one embodiment, the alkyl group has 1-12 carbons. In another embodiment, the alkyl group has 1-7 carbons. In another embodiment, the alkyl group has 1-6 carbons. In another embodiment, the alkyl group has 1-4 carbons. The alkyl group may be unsubstituted or substituted by one or more groups selected from halogen, hydroxy, alkoxy carbonyl, amido, alkylamido, dialkylamido, nitro, amino, alkylamino, dialkylamino, carboxyl, thio and thioalkyl.

A “haloalkyl” group refers to an alkyl group as defined above, which is substituted by one or more halogen atoms, e.g. by F, Cl, Br or I.

An “aryl” group refers to an aromatic group having at least one carbocyclic aromatic group or heterocyclic aromatic group, which may be unsubstituted or substituted by one or more groups selected from halogen, haloalkyl, hydroxy, alkoxy carbonyl, amido, alkylamido, dialkylamido, nitro, amino, alkylamino, dialkylamino, carboxy or thio or thioalkyl. Nonlimiting examples of aryl rings are phenyl, naphthyl, pyranyl, pyrrolyl, pyrazinyl, pyrimidinyl, pyrazolyl, pyridinyl, furanyl, thiophenyl, thiazolyl, imidazolyl, isoxazolyl, and the like.

A “hydroxyl” group refers to an OH group. An “alkenyl” group refers to a group having at least one carbon to carbon double bond. A halo group refers to F, Cl, Br or I.

An “arylalkyl” group refers to an alkyl bound to an aryl, wherein alkyl and aryl are as defined above. An example of an aralkyl group is a benzyl group.

As contemplated herein, the SARM compounds described herein include an analog, derivative, isomer, metabolite, pharmaceutically acceptable salt, pharmaceutical product, hydrate, N-oxide, impurity, prodrug, polymorph, crystal, or combinations thereof of the SARM compound.

As defined herein, the term “isomer” includes, but is not limited to, optical isomers and analogs, structural isomers and analogs, conformational isomers and analogs, and the like.

It will be appreciated by those skilled in the art that the SARM compounds described herein contain at least one chiral center. Accordingly, the SARMs may exist in, and be isolated in, optically-active or racemic forms. Some compounds may also exhibit polymorphism. It is to be understood that the compounds described herein can be any racemic, optically-active, polymorphic, or stereroisomeric form, or mixtures thereof. In one embodiment, the SARMs are the pure (R)-isomers. In another embodiment, the SARMs are the pure (S)-isomers. In another embodiment, the SARMs are a mixture of the (R) and the (S) isomers. In another embodiment, the SARMs are a racemic mixture comprising an equal amount of the (R) and the (S) isomers. It is well known in the art how to prepare optically-active forms (for example, by resolution of the racemic form by recrystallization techniques, by synthesis from optically-active starting materials, by chiral synthesis, or by chromatographic separation using a chiral stationary phase).

Pharmaceutically acceptable salts of SARM compounds are for example amino-substituted compounds with organic and inorganic acids, for example, citric acid and hydrochloric acid. Pharmaceutically acceptable salts can also be prepared from the phenolic compounds by treatment with inorganic bases, for example, sodium hydroxide. Also, esters of the phenolic compounds can be made with aliphatic and aromatic carboxylic acids, for example, acetic acid and benzoic acid esters.

The term “derivative” includes but is not limited to ether derivatives, acid derivatives, amide derivatives, ester derivatives and the like.

The term “hydrate” includes but is not limited to hemihydrate, monohydrate, dihydrate, trihydrate and the like.

The term “metabolite” means any substance produced from another substance by metabolism or a metabolic process.

The term “pharmaceutical product” means a composition suitable for pharmaceutical use (pharmaceutical composition), as defined herein.

The term “prodrug” means a substance which can be converted in-vivo into a biologically active agent by such reactions as hydrolysis, esterification, desterification, activation, salt formation and the like.

The term “crystal” means a substance in a crystalline state. The term “polymorph” refers to a particular crystalline state of a substance, having particular physical properties such as X-ray diffraction, IR spectra, melting point, and the like.

The following examples are presented in order to more fully illustrate the preferred embodiments of the invention. They should in no way be construed, however, as limiting the broad scope of the invention.

### EXPERIMENTAL DETAILS SECTION

### EXAMPLE 1

### Analytical Determination of the Concentration of Compound VII in Rat Plasma

The analytical method described herein is a liquid chromatographic-mass spectrometric (LC/MS) assay to determine the concentrations of SARM compounds, for example compound VII, in plasma.

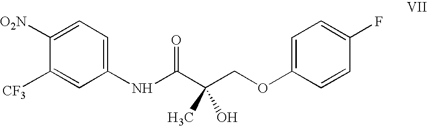

Analytical Method

Stationary Phase: monolithic column (Chromolith SpeedROD, RP-18e, 4.6′ 50 mm; Merck KgaA, Darmstadt, Germany).

Mobile Phase: A: Acetonitrile: H₂O 5:95 (0.1% acetic acid)

B: Acetonitrile: H₂O 95:5 (0.1% acetic acid)

Mobile Phase Gradient Program:

| Time(min) | B(%) |
|---|---|
| 0 | 50 |
| 5.0 | 50 |
| 5.1 | 100 |
| 7.5 | 100 |
| 7.6 | 50 |

Equilibration Time: 1.5 min

Flow rate: 1 mL/min

Column temperature: 25° C.

Instrument: Agilent System 1100 single quadrupole LC/MSD. Single-ion monitor: 014: 401.10 Internal standard (Chlorine analog): 417.10. Ionization Mode: electrospray ionization, negative mode.

Ionization Parameters:

| | |
|---|---|
| Fragmenter voltage | 180 |
| Drying gas flow | 12 mL/min |
| Nebulizer pressure | 45 psi |
| Drying gas temperature | 350° C. |
| Capillary voltage | 1500 V |

Determination of SARM Concentrations in Mammalian Plasma

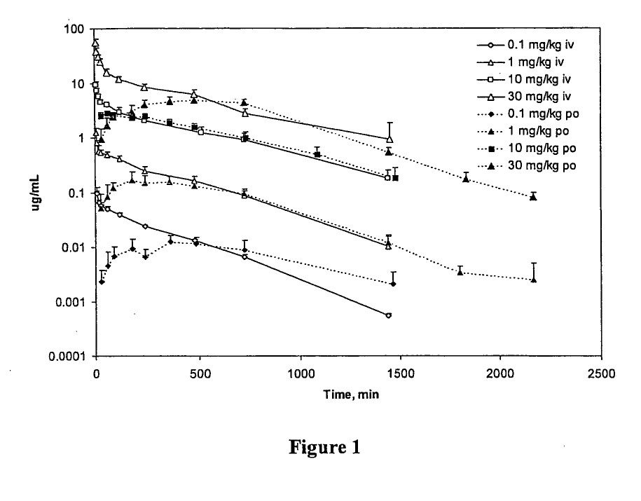

As depicted in FIG. 1, this method was used to quantify concentrations of Compound VII in rat plasma after intravenous and oral administration of the drug at doses ranging from 0.1 to 30 mg/kg.

The method is a liquid chromatographic-mass spectrometric (LC/MS) assay which utilized a closely related structural analog as an internal standard. The assay involved liquid-liquid extraction of the analytes from plasma using ethyl acetate. The resulting organic phase was evaporated to dryness under nitrogen and then reconstituted with mobile phase. Individual samples were injected onto a monolithic column and separated from interferences using a gradient mobile phase comprised of initial phase of acetonitrile-water (50:50, 0.1% acetic acid) at a flow rate of 1 mL/min.

The mass spectrometer (Agilent 1100) was programmed in the negative single-ion monitoring mode to facilitate the detection and quantification of the ions of Compound VII and the internal standard at m/z 401.10 and 417.10, respectively. The peak area ratios were linear (R²>0.999) over the concentration range 4.5-300 ng/mL. Within- and between-day precision was within 0.5 to 14% coefficient variation and the accuracy was 95.1 to 107.5% of the nominal concentrations.

The relative recoveries of Compound VII from rat plasma ranged from 90.5% to 108.1%. The use of elevated flow-rates enabled reduction of the analysis time by a factor of 3 compared to assays based on conventional narrow-bore columns.

The validated method was utilized to establish various pharmacokinetic parameters after intravenous and oral doses of Compound VII to rats.

### EXAMPLE 2

### Validation of an LC-MS-MS Method for the Quantitation of Compound VI in Human Heparinized Plasma

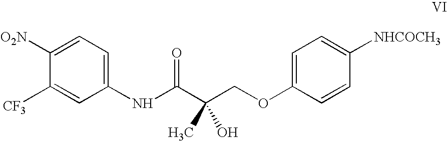

Validation Summary

Method

An LC-MS-MS method was developed and validated for the quantification of Compound VI in human heparinized plasma. Samples were spiked with internal standard (d3-Compound VI) and extracted using an organic solvent. The extracts were evaporated to dryness and reconstituted for injection onto an LC-MS-MS. The PE Sciex API 365, using an ESI interface, was employed in this study. Negative ions were monitored in the MRM (multiple reaction monitoring) mode.

Conduct of Validation

A single set of calibration standards, placed near the beginning of each batch, defined a standard curve from which 6 replicates of quality control samples at 3 concentrations were determined. Over a period of 14 days, 4 batches (V06, V07, V08, and R09) were assayed to determine the inter-batch and intra-batch reproducibility. Batch R07 was included in the database for reinjection and refrigeration stability data. Batch T04 was included in the database for sensitivity and selectivity data. Benchtop stability, freeze/thaw stability, and storage stability at −70° C. were also determined.

During the validation, quality control samples were stored in a freezer set at −70° C.

Analytical Data Treatment

Chromatograms were integrated using Analyst® software, and raw data was subsequently transferred into the OpenVMS® on AlphaServer® Systems Oracle® database. A weighted [(1/x2) where x=analyte concentration] quadratic regression was used to determine slopes, intercepts, and correlation coefficients. The resulting parameters were used to calculate concentrations:

$$Ratio = \begin{matrix}A \times {( concentration )}^{2} + \\ B \times ( concentration ) + C\end{matrix}$$

where “Ratio” is the ratio of the compound peak area to the internal standard peak area.

Extraction Recovery:

104% over a concentration range of 0.0500-200 ng/mL (Table 1), vs. 103% of internal standard (not shown):

TABLE 1
Compound VI Recovery

| | 0.0500 ng/mL | | 2.00 ng/mL | | 200 ng/mL | |
|---|---|---|---|---|---|---|
| | A | B | A | B | A | B |
| | 462 479 | 16299 | 15643 | 1160741 | | |
| | | | 1151892 | | | |
| | 390 450 | 15757 | 16667 | 1204254 | | |
| | | | 1140199 | | | |
| | 524 505 | 19982 | 15797 | 1107717 | | |
| | | | 1137737 | | | |
| | 659 435 | 15988 | 15952 | 1193838 | | |
| | | | 1180410 | | | |
| | 639 492 | 15944 | 16106 | 1234635 | | |
| | | | 1119426 | | | |
| | 564 494 | 13322 | 16043 | 1255710 | | |
| | | | 1203581 | | | |
| Mean | 540 476 | 16215 | 16035 | 1155541 | 1192816 | |
| C.V. % | 19.1 | 5.8 | 13.2 | 2.2 | 2.7 | 4.5 |
| N | 6 | 6 | 6 | 6 | 6 | 6 |
| Recovery | | 113 | | 101 | | 97 |
| % | | | | | | |

| | |
|---|---|
| Mean Recovery % | 104 |

A (EXT) = Compound VI, extracted from spiked control human heparinized plasma
B (REX) = Compound VI, spiked into extracted control human heparinized plasma

Stability

TABLE 2
Compound VI in the Biological Matrix:

| | | As Percent | |
|---|---|---|---|
| | Period | of Control | Table |
| Benchtop | 25 hrs at RT under | 87-115 | 8 |
| | white light | | |
| Freeze/Thaw | 6 cycles | 92-99 | 9 |
| Long Term | 15 days at −70° C. | 87-108 | 10 |
| Storage | | | |

TABLE 3
Compound VI in the Extracted Sample

| | | As Percent | |
|---|---|---|---|
| | Period | of Control | Table |
| Reinjection | 61 hrs | 92-120 | 7 |
| Refrigeration | 70 hrs | 92-120 | 7 |

Sensitivity and Selectivity

To demonstrate sensitivity and selectivity, 10 lots of control human heparinized plasma were assayed, and 10 out of 10 did not show interfering peaks at the retention time of the compounds of interest. Compound VI was spiked into each lot at a concentration equal to the LLOQ, and 10 out of 10 lots quantitated within 20% of the theoretical LLOQ value when back-calculated against calibrating standards. Compound VI was spiked into each lot at a concentration equal to the ULOQ, and 10 out of 10 lots quantitated within 15% of the theoretical ULOQ value when back-calculated against calibrating standards. Typical signal-to-noise ratio at 0.05 ng/mL was 8:1. Sensitivity and selectivity data are shown in Table 4.

TABLE 4
Compound VI Sensitivity and Selectivity

| | | | A1 | | A2 | | B | |
|---|---|---|---|---|---|---|---|---|
| | Blank | LLOQ | 0.0500 ng/mL | | 200 ng/mL | | 0.0500 ng/mL | |
| | peak | peak | Calc. | | Calc. | | Calc. | |
| Lot# | area | area | Conc. | % Dev | Conc. | % Dev. | Conc. | % Dev. |
| 1 | 0 | 3654 | −0.0492 | 1.6 | 195 | −2.2 | +0.0539 | 7.8 |
| 2 | 0 | 3836 | −0.0464 | 7.2 | 200 | +0.1 | +0.0553 | 10.6 |
| 3 | 0 | 4085 | +0.0566 | 13.2 | 197 | −1.1 | +0.0544 | 8.8 |
| 4 | 0 | 4318 | +0.0554 | 10.8 | 198 | −0.9 | −0.0450 | 10.0 |
| 5 | 0 | 4123 | +0.0550 | 10.0 | 213 | +6.6 | −0.0473 | 5.4 |
| 6 | 0 | 4166 | +0.0524 | 4.8 | 216 | +8.2 | −0.0464 | 7.2 |
| 7 | 0 | 4361 | +0.0531 | 6.2 | 209 | +5.0 | — | — |
| 8 | 0 | 3990 | +0.0524 | 4.8 | 205 | +3.0 | — | — |
| 9 | 0 | 4331 | +0.0557 | 11.4 | 209 | +4.5 | — | — |
| 10 | 0 | 4438 | +0.0578 | 15.6 | 214 | +7.4 | — | — |
| Mean | | | 0.0534 | | 206 | | 0.0504 | |
| C.V. % | | | 6.6 | | 3.7 | | 9.2 | |
| R.E. % | | | +6.8 | | +3.0 | | +0.8 | |
| N | | | 10 | | 10 | | 6 | |

A1 = Compound VI, 0.0500 ng/mL, 1 replicate in 10 lots of human heparinized plasma
A2 = Compound VI, 200 ng/ml, 1 replicate in 10 lots of human heparinized plasma
B = Compound VI, 0.0500 ng/ml, 6 replicates in a single lot of human heparinized plasma
—= No value, analyzed n = 6

Extraction recoveries were calculated by comparison of the peak area counts of Compound VI added prior to the extraction (EXT) to those of Compound VI added after extraction (REX) using the equation (A/B×100).

To evaluate sensitivity and selectivity, 10 lots of human heparinized plasma were spiked with Compound VI at 0 ng/mL, 0.0500 ng/mL, and 200 ng/mL. At 0.0500 ng/mL, 10 out of 10 lots quantitated within 20% of the theoretical value when back-calculated against calibration standards. At 200 ng/mL, 10 out of 10 lots quantitated within 15% of the theoretical value when back-calculated against calibration standards.

No interference at the retention time of Compound VI was shown for 10 out of 10 lots. The typical signal-to-noise ratio at 0.0500 ng/mL was 8.1:1.

### EXAMPLE 3

### Analytical Determination of the Concentration of Compound VI in Rat Plasma

The analytical method described herein is an HPLC/UV assay to determine the concentrations of SARM compounds, for example compound VI, in plasma.

Summary

Applicants have developed and validated a gradient HPLC method for the quantitation of Compound VI in rat plasma. Validation studies were performed to demonstrate method accuracy, linearity, precision, sensitivity, and selectivity. The gradient HPLC method for Compound VI in rat plasma is valid over the range of 0.8 μg/mL to 16.2 μg/mL. Compound A was validated as an internal standard for this assay.

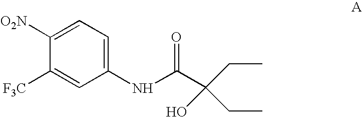

The lower limit of detection was characterized at 0.4 μg/mL, which is two-fold lower than the validated dynamic range of the assay. Conditions for solution stability were defined for up to 46 days after initial preparation. Stability of the analyte in extracted plasma matrix was demonstrated for up to 72 hours post-extraction. Extracted samples were stable for a minimum of three freeze/thaw cycles with no evidence of degradation. Samples which originally tested “above-the-limit” of the standard curve were validated for quantitation using either a 1:5 or a 1:10 dilution with blank plasma prior to extraction. Extraction efficiency (recovery) was found to be approximately 61%.

Introduction

Applicants have developed a gradient HPLC method for the analysis of Compound VI in rat plasma. In the present study, method validation was performed to demonstrate accuracy, linearity, precision, and sensitivity over the dynamic range of the method (0.8 μg/mL to 16.2 μg/mL). A lower limit of detection was evaluated. Other method parameters tested included extraction efficiency, suitability of aqueous solutions, evaluation of standards, stability of aqueous solutions and extracted samples, stability of analyte in extracted sample matrix over a minimum of three freeze/thaw cycles, specificity of analyte and internal standard in sample matrix, and quantitation of samples that are above-the-limit of the assay range. Inter-instrument comparison was also made to demonstrate intermediate precision of the method.

Methods

Extraction Procedure:

Rat plasma samples were spiked with Compound VI and internal standard and then extracted using a liquid/liquid extraction technique. Quantitation was performed on a Waters Alliance HPLC system with UV detection. Standard curves were produced by linear regression using the peak area ratios of analyte to internal standard and a 1/y curve weighting.

| | |
|---|---|
| Assay Type: | HPLC-UV |
| Analyte: | Compound VI |
| Internal Standard: | Compound A |
| Matrix: | Rat Plasma |
| Volume per Analysis: | 0.25 mL |
| Extraction: | Liquid/Liquid |
| Calibration Range: | 0.8 to 16.2 μg/mL |

HPLC Instrument Conditions:

| | |
|---|---|
| Column: | Symmetry C-8 (3.9 × 150 mm) |
| | equipped with equivalent |
| | guard column |
| Injection Volume: | 50 μL |
| Flow Rate: | 1.0 mL/min. |
| Column Temperature: | 40° C. |
| Detection: | UV; 260 nm |
| Mobile Phase: | Reservoir A: Neat HPLC Acetonitrile |
| (Mixed at pump head) | Reservoir B: HPLC grade water |

Gradient Program:

| Time | % A | % B |
|---|---|---|
| 0 | 40 | 60 |
| 1 | 40 | 60 |
| 2 | 40 | 60 |
| 5 | 50 | 50 |
| 12 | 50 | 50 |
| 15 | 40 | 60 |

Results and Discussion

Linearity:

Calibration curves were constructed by spiking plasma with aqueous standards to prepare plasma concentrations in the range of 0.8 to 16.2 μg/mL. All calibration curves had a correlation coefficient of 0.99 or greater over the standard range of 0.8 to 16.2 μg/mL.

Sensitivity:

A limit of detection (LOD) was defined at 0.4 μg/mL with a peak that was discrete and identifiable, with good precision (8.1% relative standard deviation) and acceptable accuracy (80.8% of nominal). The LOD standard is two-fold below the lower limit of quantitation level established in this assay. The peak at the lower limit of quantitation (0.8 μg/mL) was identifiable, discrete, and reproducible with a precision of 7.1% and an accuracy of 97.3% when quantitated based on internal standard (shown in Table 1). The precision and accuracy of the lowest controls (LLQC, 0.8 μg/mL) were 6.4% and 103.3%, respectively.

Quality Control Samples:

Quality control samples were prepared by diluting aqueous stock solutions in blank rat plasma screened for interference. First, an aqueous solution (approximately 40 μg/mL) of Compound VI was prepared by transferring 2 mL of Compound VI control stock (approximately 200 μg/mL) to 10 mL volumetric flask and bringing to volume with 40% acetonitrile. The concentration of the control stock solution was tested against an aqueous standard curve and the actual concentration of the solution was found to be 230.8 μg/mL. The diluted stock was 46.2 μg/mL. This aqueous solution was used to prepare quality control solutions as follows:

High Quality Control—Nominal concentration: 13.85 μg/mL^{$}

Medium Quality Control—Nominal concentration: 4.62 μg/mL^{$}

Low Quality Control—Nominal concentration: 2.77 μg/mL^{$}

Lower Limit Quality Control—Nominal concentration: 0.92 μg/mL^{$}. ^{$}It should be noted that the actual concentrations differ from the protocol nominal concentration slightly due to a minor difference in the stock concentration. The calculations for quality control samples for this body of work will be based on the nominal concentrations reported here, unless indicated otherwise.

These concentrations cover the range of calibration used to prepare standard curves for quantitation of Compound VI as suggested by the “Guidance for Industry for Bioanalytical Method Validation” from the Food and Drug Administration's (FDA) Center for Drug Evaluation and Research (CDER) published in May of 2001.

The quality control samples were extracted and analyzed with the calibration standard curve range of 0.8 to 16.2 μg/mL. When assayed, the mean calculated value was divided by the nominal concentration and multiplied by 100 to generate the “percent of nominal”. The results demonstrated a mean percent of nominal of 100.3, 90.5, 99.3, and 103.3 for the high, medium, low, and lower limit quality control samples, respectively.

“Above-the-Limit” Quality Control Sample:

The ability to dilute samples originally found to be above the upper limit of the standard curve was tested by preparing “Above-the-limit” (ATL) quality control samples of rat plasma spiked with aqueous standards (nominal concentration of 46.2 μg/mL) and then diluting before analysis. Two dilution methods were tested. The first dilution method involved diluting the spiked quality control sample with “blank” plasma at 1:5 and 1:10 ratio prior to extracting the samples. The second dilution method involved extracting the “above-the-limit” quality control sample as per standard procedure, and then increasing the reconstitution volume (diluting the extract) at 1:5 or 1:10 ratio prior to analysis. Results from diluting samples with blank plasma prior to extraction demonstrated an accuracy of 112.6 and 113.5% and a precision of 3.9 and 4.5% for the 1:5 and 1:10 dilutions, respectively. For ATL samples that were analyzed after diluting the final extracts in 1:5 and 1:10 ratios, the accuracy was 103.1 and 100.1% of nominal with a precision of 1.9 and 2.4% RSD, respectively.

Internal Standard Concentration:

The internal standard spiking solution concentration at 80 μg/mL was determined to be acceptable after comparison with the aqueous calibration spiking standard at 40 μg/mL. Acceptance criteria stated that the concentration of the internal standard spiking solution should result in an instrument response ±20% of the highest calibration standard contained in the standard curve range. The mean area under the curve (instrument response) for the 40 μg/mL calibration standard was 4028196 and the mean area under the curve for the internal standard spiking solution (80 μg/mL) was 3404461. Thus, at 80 μg/mL, the internal standard produced an instrument response that was 15.5% less than the highest calibration standard (40 μg/mL).

Accuracy and Precision:

Within Day (IntraDay) and Between Day (InterDay) accuracy and precision were established by analyzing four replicate QC samples at four different levels on three separate days. The relative standard deviation (RSD) and the calculated versus nominal (percent of nominal) concentrations were used to determine acceptable precision and accuracy for the method.

IntraDay precision was demonstrated where the mean quality control concentration for the high, medium, low, and low-low controls for each of the three days ranged from 0.6 to 11.9% RSD, with a mean RSD of 3.3% . The percent of nominal for the controls on each day was used to establish IntraDay accuracy. The mean quality control concentration for each level was within 20% of nominal on each day as indicated in Table 7B. The highest deviation from nominal was in the day 1 LLQC sample with only 81.6% accuracy, and the average accuracy at all levels for all days was found to be 94.2%.

Acceptable InterDay (Between Day) precision and accuracy were also demonstrated for the Compound VI Val-ANA-081502-01 method, as shown in Table 7C. Precision was good, where the mean quality control concentrations for HQC, MQC, LQC, and LLQC between days exhibited RSD of 1.5, 2.8, 3.4, and 7.7%, respectively. InterDay accuracy was acceptable at all levels, with the greatest InterDay inaccuracy found in the LLQC having a deviation from nominal of 11.0% (89.0% accuracy).

Inter-Instrument Comparison (Instrument Qualification):

A calibration curve was generated on each of two Alliance HPLC instruments (Instrument #1007 and #1011) in the Analytical Support Laboratory using the same extracted calibration standards and matching instrumental set-up and conditions.

The curves generated by both instruments were linear across the range of calibration standards (0.8 to 16.2 μg/mL). After regression analysis of the linear curve, the correlation coefficients were found to be 0.999845 and 0.999883 and the slopes were 0.942 and 0.997 for instruments 1007 and 1011, respectively. All standard levels had a mean percent deviation from nominal of 15% or less (the greatest mean percent deviation from nominal was on instrument 1007 in the level 1 standard at 4.72%). Results also indicate that instruments were equivalent in determining accurate concentrations, where for both instruments actual concentrations for all standards were within 1.7% of nominal. The low and high calibration standards were within the pre-determined 20% of nominal on both instruments. Percent interference at the retention time for Compound VI analyte in the blank standard versus the level 1 standard (lower limit of quantitation, LLOQ) was zero. These results demonstrate that both systems are qualified to generate data for the method of determining Compound VI concentration in rat plasma extracts.

Stability:

The stability of Compound VI samples under various conditions was evaluated.

Three Freeze/Thaw (F/T) cycles for frozen samples: Control samples were prepared and stored at approximately −80° C. for between 24 and 168 hours. Samples were subjected to three cycles of re-freezing and thawing prior to being extracted and analyzed. The results demonstrate the stability of the samples. Change from initial (% degradation) was −1.02%, 0.16%, 3.91%, and −3.57% for the high, medium, low and low-low controls, respectively.

72 hour extract stability at ambient: Three sets of control samples were extracted and reconstituted into WISP vials for injection. One set was injected immediately (0 hr), one set after 24 hours at room temperature and another set approximately 72 hours post-extraction. Calibration curves were consistent with the timed and fresh extracts, with no apparent change in linearity as slope was constant with only a −1.4% difference over 72 hour period, and a correlation coefficient of approximately 0.999 for all three timepoints evaluated. Individual calibrators remained suitable for constructing a standard curve to quantitate Compound VI, with no apparent increased variability over the elapsed 72 hours (mean percent deviation from nominal≦7.5% at all levels). The maximum percent change (difference from initial) for the calibrators was 4.6% from initial.

In addition to the finding that the calibration curves remained suitable for quantitating samples over the elapsed 72 hour time course, the results indicate that the quality control extracts were stable up to 72 hours at room temperature prior to being analyzed (not shown). Inspection of the chromatograms did not reveal any artifact or degradation which would lead to the conclusion that the analytes were unstable in the biological matrix.

In conclusion, all of the 72 hour samples had acceptable change with a maximum of 9.3% from initial. Because there was no loss in amount of analyte calculated for the 72 hour samples, and because the calibration standards and controls were stable after 72 hours at room temperature, the findings at 24 hours were not concluded to indicate instability of control extracts at 24 hours.

6 hour stability of analyte in sample matrix at ambient (pre-extraction): Blank plasma was spiked with Compound VI with internal standard and one portion extracted immediately with additional portions being assayed 1 hour, 2 hours, 4 hours, and 6 hours after preparation. Pre-extracted samples demonstrated stability of analyte in the rat plasma, where the mean difference from initial for all timed samples was −3.5%, with the greatest difference at −7.5% in the 6 hour high control sample.

Stability of aqueous standard solutions at room temperature and 4° C.: Aqueous standard solutions were injected at initial preparation (0 hour) and then again at various intervals post-preparation. Samples were transferred from glass volumetric flasks and stored in polypropylene tubes at room temperature over a 72-hour period to evaluate room temperature stability and over 29 day period for refrigerated stability study.

The 0 hour sample demonstrated good chromatography with only three principal peaks at the expected retention times for Compound VI analyte, cmII87, and internal standard. After 24 hours at room temperature, there was a slight increase in the baseline of the chromatogram at multiple retention times. The room temperature 72-hour injection demonstrated similar peak pattern in the chromatogram as the 24-hour injection, with even greater increase for the newly apparent peaks in the baseline). Interestingly, despite an increase in the baseline with the new peaks, there was not a significant corresponding change (p<0.05) in the peak area for Compound VI over the 72-hour time course (less than 2.23% difference from initial at all levels). These results indicate that while the individual components were not degraded over the 72-hour time period, new peaks arose in the solutions. These peaks might be attributed to bacterial growth or plasticizers leaching out of the polypropylene tubing into the organic phase of the diluent.

Three levels of aqueous standard solutions prepared for validation were likewise tested for stability under refrigerated conditions (2-6° C.) for a period of 29 days from initial mixing. After 7 days of storage in plastic containers at 4° C., several peaks appeared in the baseline that were not present in the chromatograms at initial test. The new peaks were chromatographically similar in size and retention time to those observed in solutions stored in plastic containers for a 72-hour period (at room temperature). Importantly, they did not interfere at the retention times for Compound VI, cmII87 (system suitability), or internal standard. Aside from the newly apparent peaks in the baseline, at day 7 the mixture components were stable with a maximum percent of degradation of 1.4 and 4.5 observed for Compound VI and internal standard, respectively. Solutions were again tested for 4° C. stability on days 14, 22, and 29. On days 14 and 22, the Compound VI and internal standard analytes in the mixed solutions stored at 4° C. were not degraded (percent degradation less than 2.8 and 6.5, respectively). There was a 10.9% change from initial for the cmII87 in only the high system suitability mixure on day 22, which is slightly outside the protocol specified 10% range, however when that solution was tested at a later date (d29), the cmII87 was stable, with only a 2.3% change from initial. One of the new peaks arising in the solution at approximately 7.5 minutes retention time chromatographed very close to cmII87. Despite this, cmII87 could still be baseline resolved in the presence of this peak. It was noted that the same mixtures stored in glass containers at 4° C. (for system suitability) did not have the newly arising peaks. Thus, it seems the peaks were a product of the polypropylene storage containers and not degradation of the analytes.

After 29 days of storage, the internal standard was exhibiting a diminished response in the low level mixture (decreased by 18.1% for the LSS sample). Interestingly, while the internal standard in low aqueous solution was decreased, in the medium and high aqueous standards remained stable (percent degradation of 6.6 and 4.0, respectively). Compound VI analyte remained stable over the 29-day period (maximum percent degradation of 5.5 in the medium aqueous standard at day 29). Similar to Compound A, the cmII87 analyte was changed out of the specified range of 10% from initial on day 29 with a % degradation of 12.4%.

Single-component aqueous stock solutions of Compound VI and Compound A in 40% acetonitrile diluent were injected on day 0 (initial) and again on day 46. Results indicate that when these solutions are stored at 4° C. in glass containers for over a 46-day period they are stable and chromatographically suitable with no significant interfering peaks, and minimal degradation (2.53 and 8.58% for Compound VI and Compound A, respectively).

In summary, mixed solutions of Compound VI, cmII87 and internal standard in 40% acetonitrile diluent are stable for up to 72-hous at room temperature and for between 22 and 29 days at 4° C. Some solutions demonstrated contaminating peaks, however all mixture components were baseline resolved in the presence of the contaminants. Since the peaks were present in solutions stored in polypropylene at room temperature and 4° C., they are more likely a product of plasticizer than bacterial growth. Solutions of single-component analytes are stable for at least 46 days when stored at 4° C. in glass containers. Thus, the ideal storage condition for all solutions was determined to be at 4° C. in glass containers. Even when stored at ideal, mixed solutions should not be used beyond 29 days after preparation.

Selectivity (Specificity):

Six individual blank plasma sources from Sprague-Dawley rats were carried through the assay procedure to demonstrate selectivity. The chromatograms from each of the six sources were similar to that of the pooled blank Sprague-Dawley plasma screened prior to preparation of quality control samples. No peaks were observed in the region of Compound VI in the blank plasma.

Extraction Efficiency:

Extraction efficiency of Compound VI from the low, medium, and high controls was 60.0%, 59.2%, and 64.7%, respectively. The mean extraction efficiency for Compound VI in rat plasma was found to be 61.3%. The extraction of the internal standard from rat plasma at 65.2% efficiency was approximately the same as for the analyte.

Conclusion

A sensitive and specific gradient HPLC assay with UV detection for the determination of Compound VI in rat plasma has been validated for the concentration range of 0.8 to 16.2 μg/mL. Validation studies were performed to demonstrate method accuracy, linearity, precision, sensitivity, and selectivity. Compound A was validated as an internal standard for this assay. The lower limit of detection was characterized at 0.4 μg/mL, which is two-fold lower than the validated dynamic range of the assay. Conditions for solution stability were defined for up to 46 days after initial preparation. Stability of the analyte in extracted plasma matrix was demonstrated for up to 72 hours post-extraction. Extracted samples were stable for a minimum of three freeze/thaw cycles with no evidence of degradation. A method to quantitate samples which originally test “above-the-limit” of the standard curve was validated using either a 1:5 or a 1:10 dilution with blank plasma prior to extraction. Extraction efficiency (recovery) was found to be approximately 61%.

### EXAMPLE 4

### Analytical Determination of the Concentration of Compound VI in Dog Plasma

Summary

Applicants have developed an analytical method using HPLC for the quantitation of SARMs, e.g. Compound VI, in plasma. The method was validated in dogs, demonstrating method accuracy, selectivity, sensitivity, stability, and recovery of the bioanalytical method using dog plasma as the biological matrix. Sample stability in rat plasma has also been addressed. The current studies extend the already validated bioanalytical method for a change of species (rat to dog) within the biological matrix.

These results demonstrate that the gradient HPLC method is valid for analysis of Compound VI in dog plasma matrix over the concentration range of 0.8 μg/mL to 16.0 μg/mL. The analyte Compound A was validated as an internal standard for this assay. Stability of the analyte in extracted plasma matrix was demonstrated for up to 72 hours post-extraction. Quality control samples were stable for six hours pre-extraction at room temperature, and a minimum of three freeze/thaw cycles with no evidence of degradation. Extraction efficiency (recovery) for Compound VI in dog plasma was approximately 77% for Compound VI, and 82.7% for internal standard.

Introduction

Applicants have developed a gradient HPLC method for the analysis of Compound VI in plasma. In the present study, a partial method validation was performed to demonstrate accuracy, linearity, precision, selectivity, sensitivity over the dynamic range of the method (0.8 μg/mL to 16.0 μg/mL). Other method parameters tested included extraction efficiency, stability of extracted samples up to 72 hours, stability of analyte in plasma matrix over a minimum of three freeze/thaw cycles, and specificity of analyte and internal standard in sample matrix.

Methods

Extraction Procedure

Dog plasma samples were spiked with Compound VI and internal standard and then extracted using a liquid/liquid extraction technique. Quantitation was performed on a Waters Alliance HPLC system with UV detection. Standard curves were produced by linear regression using the peak area ratios of analyte to internal standard and a 1/y curve weighting.

Assay Conditions:

| | |
|---|---|
| Assay Type: | HPLC-UV |
| Analyte: | Compound VI |
| Internal Standard: | ck1-149 |
| Matrix: | Dog Plasma |
| Volume per Analysis: | 0.25 mL |
| Extraction: | Liquid/Liquid |
| Calibration Range: | 0.8 to 16.0 μg/mL |

HPLC Instrument Conditions:

| | |
|---|---|
| Column: | Symmetry C-8 (3.9 × 150 mm) |
| | equipped with equivalent guard |
| | column |
| Injection Volume: | 50 μL |
| Flow rate: | 1.0 mL/min. |
| Column Temperature: | 40° C. |
| Detection: | UV; 260 nm |
| Mobile Phase: | Reservoir A: Neat HPLC Acetonitrile |
| (Mixed at pump head) | Reservoir B: HPLC grade water |

Gradient Program:

| Time | % A | % B |
|---|---|---|
| 0 | 40 | 60 |
| 1 | 40 | 60 |
| 2 | 40 | 60 |
| 5 | 50 | 50 |
| 12 | 50 | 50 |
| 15 | 40 | 60 |

Compound A—Internal Standard

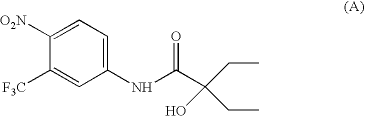

Results and Discussion

Linearity:

Calibration curves were constructed by spiking plasma with aqueous standards to prepare plasma concentrations in the range of 0.8 to 16.0 μg/mL. All calibration curves had a correlation coefficient of 0.999 or greater over the standard range of 0.8 to 16.0 μg/mL.

Sensitivity:

The slope of the line generated by linear regression of the data is a function of sensitivity. (In the “Guidance for Industry for Bioanalytical Method Validation” from the Food and Drug Administration's (FDA) Center for Drug Evaluation and Research (CDER) published in May of 2001). Hence, the slope of calibration curve in the current study utilizing dog plasma matrix is compared to the previous validation in rat plasma matrix as an indication of sensitivity. In the present study, the mean slope for all calibration curves was 0.980 (for the approximate standard range of 0.8 to 16.0 μg/mL). The mean slope for all calibration curves generated during the validation of this assay in rat plasma was 0.964 (ref validation report). Transformation of the data reveals that the sensitivity of the assay with dog plasma as a matrix is equivalent (0.980/0.964×100=101.7%) to the validated rat method. In addition, the sensitivity of the lowest calibration standard was tested and the results indicate that the LLOQ standard produced an instrument response that was at least 5 times the response of blank extract, meeting the acceptance criteria specified in the testing protocol.

Further, the chromatography of the lower limit of quantitation (0.8 μg/mL) standard was evaluated. The peak at the lower limit of quantitation was identifiable, discrete, and reproducible with a precision of 11.1%. The mean precision and accuracy of the low control (LQC, 2.0 μg/mL) were 1.1% and 99.9%, respectively.

Quality Control Samples:

Quality control samples were prepared by diluting aqueous stock solutions in blank dog plasma (previously screened for interference). First, an aqueous solution (approximately 40 μg/mL) of Compound VI was prepared by transferring 2 mL of Compound VI control stock (approximately 200 μg/mL) to 10 mL volumetric flask and bringing to volume with 40% acetonitrile. The concentration of the control stock solution was tested against an aqueous standard curve and the actual concentration of the solution was found to be 219.10 μg/mL. The diluted stock was 43.81 μg/mL. This aqueous solution was used to prepare quality control solutions.

High Quality Control—Nominal concentration: 13.15 μg/mL^{$}

Medium Quality Control—Nominal concentration: 4.38 μg/mL^{$}

Low Quality Control—Nominal concentration: 2.68 μg/mL^{$}

Lower Limit Quality Control—Nominal concentration: 0.88 μg/mL^{$}. ^{$}It should be noted that the actual concentrations differ from the protocol nominal concentration slightly due to a minor difference in the stock concentration. The calculations for quality control samples for this body of work will be based on the nominal concentrations reported here, unless indicated otherwise.

These concentrations cover the range of calibration used to prepare standard curves for quantitation of Compoun VI as suggested by the “Guidance for Industry for Bioanalytical Method Validation” from the Food and Drug Administration's (FDA) Center for Drug Evaluation and Research (CDER) published in May of 2001.

The quality control samples were extracted and analyzed with the calibration standard curve range of 0.8 to 16.0 μg/mL. When assayed, the mean calculated value was divided by the nominal concentration and multiplied by 100 to generate the “percent of nominal”. The results demonstrated a mean percent of nominal of 91.9, 93.8, and 99.9 for the high, medium, and low quality control samples, respectively.

Stability:

The stability of Compound VI samples under various conditions was evaluated.

Three Freeze/Thaw (F/T) cycles for frozen samples: Control samples were prepared and stored at approximately −80° C. for between 24 and 168 hours. Samples were subjected to three cycles of re-freezing and thawing prior to being extracted and analyzed. The results demonstrate the stability of the samples. Percent change from initial (% degradation) was 3.1%, 0.2%, and 9.6% for the high, medium, and low quality control samples, respectively.

72 hour extract stability at ambient: Two sets of control samples were extracted and reconstituted into WISP vials for injection. One set was injected immediately (0 hr), the additional set was injected after 72 hours at room temperature. Calibration curves were consistent with the timed and fresh extracts, with no apparent change in linearity as slope was constant with only a 0.74% difference from initial over the 72-hour period, and a correlation coefficient of approximately 0.999 for both timepoints evaluated. Individual calibrators remained suitable for constructing a standard curve to quantitate Compound VI, with no apparent increased variability over the elapsed 72 hours (mean percent deviation from nominal≦1.2% at all levels).

In addition to the finding that the calibration curves remained suitable for quantitating samples over the elapsed 72 hour time course, the results indicate that the quality control extracts were stable up to 72 hours at room temperature prior to being analyzed, where all levels met the acceptance criteria for quality control samples over the 72-hour time period. The greatest % difference was 0.8% different from initial.

6 hour stability of analyte in sample matrix at ambient (pre-extraction): Blank plasma was spiked with Compound VI and with internal standard, with a portion being extracted immediately and the additional portion being assayed 6 hours after preparation. Pre-extracted-samples demonstrated stability of analyte in the dog plasma, where the mean % change from initial for all timed samples was −2.4%, with the greatest difference at −4.9% in the 6 hour low control sample.

Selectivity (Specificity):

Six individual blank plasma sources from beagle dogs were carried through the assay procedure to demonstrate selectivity. Two of the six sources exhibited a minor interference (4.0 and 4.1% for sources 1 and 6, respectively) at the retention time of Compound VI in the blank plasma. The same two sources also exhibited a minor interference (0.4 and 0.2 % for sources 1 and 6, respectively) at the retention time for internal standard. All other sources were free from interference in region of internal standard and analyte.

Extraction Efficiency:

Six Extraction efficiency of Compound VI from the low, medium, and high controls was 77.6%, 73.8%, and 79.0%, respectively. The mean extraction efficiency for Compound VI in dog plasma was found to be 76.8%. The extraction efficiency of the internal standard from dog plasma was demonstrated at 82.7%.

Conclusion:

A sensitive and specific gradient HPLC assay with UV detection for the determination of Compound VI in dog plasma was validated for the concentration range of 0.8 to 16.0 μg/mL. Validation studies demonstrated method accuracy, linearity, precision, sensitivity, and selectivity. The analyte ck1-149 was validated as an internal standard for this assay. Stability of the analyte in extracted plasma matrix was demonstrated for up to 72 hours post-extraction. Quality control samples were stable for six hours pre-extraction at room temperature, and a minimum of three freeze/thaw cycles with no evidence of degradation. Extraction efficiency (recovery) for Compound VI in dog plasma was approximately 77% for Compound VI, and 82.7% for internal standard.

It will be appreciated by a person skilled in the art that the present invention is not limited by what has been particularly shown and described hereinabove. Rather, the scope of the invention is defined by the claims that follow:

What is claimed is:

1. A method for detecting, identifying characterizing, purifying, obtaining structural information on, qualifying, screening, separating, fractionating and/or quantifying a Selective Androgen Receptor Modulator (SARM) compound in a sample, said method comprising the steps of:
obtaining said sample; and
detecting the presence of said SARM compound in said sample;
thereby identifying said SARM compound in said sample.

2. The method according to claim 1, wherein said sample is a biological sample.

3. The method according to claim 1, wherein said sample is a blood serum sample, a plasma sample, a urine sample, a CSF sample, a saliva sample, a fecal sample, an isolated or precipitated fraction, a protein adduct, or a protein extract.

4. The method according to claim 1, wherein said detection step comprises subjecting an aliquot from said sample to mass spectroscopy (MS), MS-MS, UV, IR, NMR, fluorescence, radiochemical detection, electrochemical detection, chemiluminscent detection, evaporative light scatter detection (ESLD), hyphenated techniques or methods, or any combination thereof.

5. The method according to claim 1, wherein the detection step comprises measuring the UV absorbance of said SARM compound.

6. The method according to claim 1, wherein the detection step comprises measuring the molecular ion peak of said SARM compound by mass spectrometry (MS).

7. The method according to claim 6, wherein said mass spectrometry is negative ion mass spectrometry.

8. The method according to claim 1, further comprising the step of determining the concentration of said SARM in said sample by comparing the amount obtained from said sample with a reference sample comprising known amounts of a reference SARM compound.

9. The method according to claim 1, further comprising the step of subjecting said sample to a chromatographic separation prior to said detection step.

10. The method according to claim 9, wherein said chromatographic separation is by liquid chromatography (LC), High Performance Liquid Chromatography (HPLC), Thin Layer Chromatography (TLC), capillary electrophoresis (CE), microLC electrophoresis, nano LC electrophoresis, gel electrophoresis (GE), isoelectric focusing gel electrophoresis, or by sample concentration.

11. The method according to claim 9, wherein said chromatographic separation is by liquid chromatography (LC), and said detection step comprises measuring the molecular ion peak of said SARM compound by mass spectrometry (MS).

12. The method according to claim 9, wherein said chromatographic separation is by High Performance Liquid Chromatography (HPLC), and said detection step comprises measuring the UV absorbance of said SARM compound.

13. The method according to claim 9, wherein said chromatographic separation is by liquid chromatography (LC), and said detection step comprises measuring the molecular ion peak of said SARM compound by MS-MS.

14. The method according to claim 9, wherein said chromatographic separation is by capillary electrophoresis (CE), and said detection step comprises measuring the molecular ion peak of said SARM compound by mass spectrometry (MS).

15. The method according to step 9, wherein the chromatographic separation comprises producing a chromatograph of said sample, said chromatograph comprising a series of peaks representing individual chemical compounds contained in said sample.

16. The method according to claim 15, further comprising the step of automatically collecting individual chemical compounds corresponding to peaks of said chromatograph into separate tubes.

17. The method according to claim 16, further comprising the step of detecting the presence of said SARM compound in an aliquot from each tube.

18. The method according to claim 17, wherein said detection step comprises subjecting each aliquot to mass spectroscopy (MS), MS-MS, UV, IR, NMR, fluorescence, radiochemical detection, electrochemical detection, chemiluminscent detection, evaporative light scatter detection (ESLD), hyphenated techniques or methods, or any combination thereof.

19. The method according to claim 17, wherein said detection step comprises measuring the UV absorbance of said SARM compound.

20. The method according to claim 17, wherein said detection step comprises measuring the molecular ion peak of said SARM compound by mass spectrometry.

21. The method according to claim 20, wherein said mass spectrometry is negative ion mass spectrometry.

22. The method according to claim 17, further comprising the step of determining the concentration of said SARM in each aliquot by comparing the amount of SARM obtained from said aliquot with a reference sample comprising known amounts of a reference SARM compound.

23. The method according to claim 1, wherein said SARM compound is a compound represented by the structure of formula I and/or its analog, derivative, isomer, metabolite, pharmaceutically acceptable salt, pharmaceutical product, hydrate, N-oxide, impurity, prodrug, polymorph, crystal, or any combination thereof 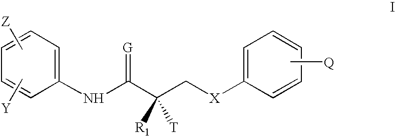
wherein G is O or S;
X is a bond, O, CH₂, NH, Se, PR, NO or NR;
T is OH, OR, —NHCOCH₃, or NHCOR
Z is NO₂, CN, COOH, COR, NHCOR or CONHR;
Y is CF₃, F, I, Br, Cl, CN, CR₃ or SnR₃;
Q is alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, SCN, NCS, OCN, NCO; or Q together with the benzene ring to which it is attached is a fused ring system represented by structure A, B or C: 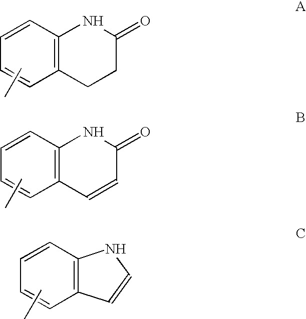
R is alkyl, haloalkyl, dihaloalkyl, trihaloalkyl, CH₂F, CHF₂, CF₃, CF₂CF₃, aryl, phenyl, halogen, alkenyl or OH; and
R₁ is CH₃, CH₂F, CHF₂, CF₃, CH₂CH₃, or CF₂CF₃.

24. The method according to claim 1, wherein said SARM compound is a compound represented by the structure of formula II and/or its analog, derivative, isomer, metabolite, pharmaceutically acceptable salt, pharmaceutical product, hydrate, N-oxide, impurity, prodrug, polymorph, crystal, or any combination thereof 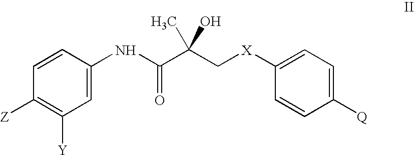
wherein X is a bond, O, CH₂, NH, Se, PR, NO or NR;
Z is NO₂, CN, COOH, COR, NHCOR or CONHR;
Y is CF₃, F, I, Br, Cl, CN, CR₃ or SnR₃;
Q is alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, SCN, NCS, OCN, NCO; or Q together with the benzene ring to which it is attached is a fused ring system represented by structure A, B or C: 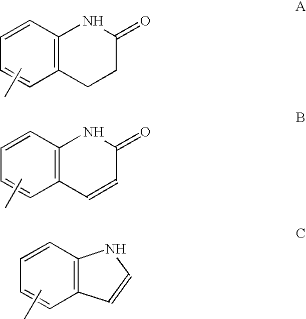
R is alkyl, haloalkyl, dihaloalkyl, trihaloalkyl, CH₂F, CHF₂, CF₃, CF₂CF₃, aryl, phenyl, halogen, alkenyl or OH.

25. The method according to claim 1, wherein said SARM compound is a compound represented by the structure of formula III and/or its analog, derivative, isomer, metabolite, pharmaceutically acceptable salt, pharmaceutical product, hydrate, N-oxide, impurity, prodrug, polymorph, crystal, or any combination thereof 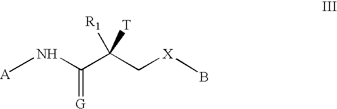
wherein X is a bond, O, CH₂, NH, Se, PR, NO or NR;
G is O or S;
R₁ is CH₃, CH₂F, CHF₂, CF₃, CH₂CH₃, or CF₂CF₃;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, halogen, alkenyl or OH;
A is a ring selected from: 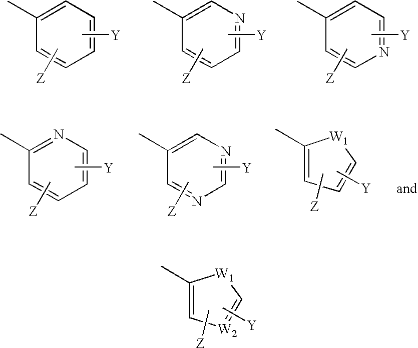
B is a ring selected from: 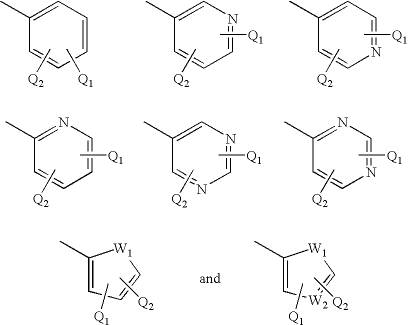
wherein A and B cannot simultaneously be a benzene ring;
Z is NO₂, CN, COOH, COR, NHCOR or CONHR;
Y is CF₃, F, I, Br, Cl, CN CR₃ or SnR₃;
Q₁ and Q₂ are independently of each other a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, SCN, NCS, OCN, NCO, 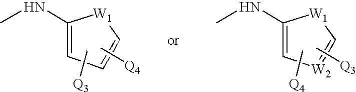
Q₃ and Q₄ are independently of each other a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R or SR, SCN, NCS, OCN, NCO;
W₁ is O, NH, NR, NO or S; and
W₂ is N or NO.

26. The method according to claim 1, wherein said SARM compound is a compound represented by the structure of formula IV and/or its analog, derivative, isomer, metabolite, pharmaceutically acceptable salt, pharmaceutical product, hydrate, N-oxide, impurity, prodrug, polymorph, crystal, or any combination thereof 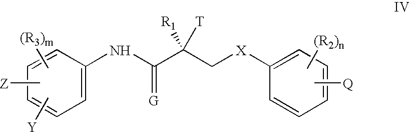
wherein X is a bond, O, CH₂, NH, Se, PR, NO or NR;
G is O or S;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
R₂ is F, Cl, Br, I, CH₃, CF₃, OH, CN, NO₂, NHCOCH₃, NHCOCF₃, NHCOR, alkyl, arylalkyl, OR, NH₂, NHR, NR₂, SR, SCN, NCS, OCN, NCO;
R₃ is F, Cl, Br, I, CN, NO₂, COR, COOH, CONHR, CF₃, SnR₃, or R₃ together with the benzene ring to which it is attached forms a fused ring system represented by the structure: 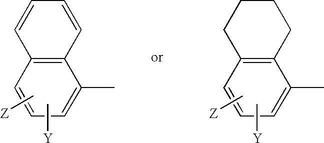
Z is NO₂, CN, COR, COOH, or CONHR;
Y is CF_{3,} F, Br, Cl, I, CN, or SnR₃;
Q is H, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OH, OR, COR, OCOR, OSO₂R, SO₂R, SR; or Q together with the benzene ring to which it is attached is a fused ring system represented by structure A, B or C: 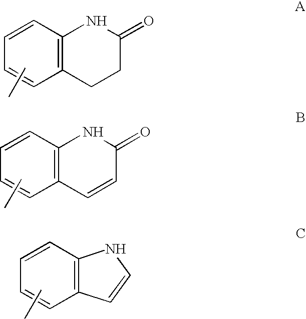
n is an integer of 1-4; and
m is an integer of 1-3.

27. The method according to claim 1, wherein said SARM compound is a compound represented by the structure of formula V and/or its analog, derivative, isomer, metabolite, pharmaceutically acceptable salt, pharmaceutical product, hydrate, N-oxide, impurity, prodrug, polymorph, crystal, or any combination thereof 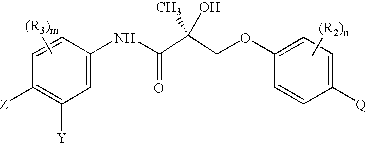
wherein
R₂ is F, Cl, Br, I, CH₃, CF₃, OH, CN, NO₂, NHCOCH₃, NHCOCF₃, NHCOR, alkyl, arylalkyl, OR, NH₂, NHR, NR₂, SR;
R₃ is F, Cl, Br, I, CN, NO₂, COR, COOH, CONHR, CF₃, SnR₃, or R₃ together with the benzene ring to which it is attached forms a fused ring system represented by the structure: 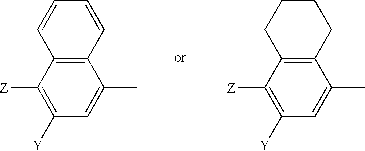
R is alkyl, haloalkyl, dihaloalkyl, trihaloalkyl, CH₂F, CHF₂, CF₃, CF₂CF₃, aryl, phenyl, halogen, alkenyl or OH;
Z is NO₂, CN, COR, COOH, or CONHR;
Y is CF₃, F, Br, Cl, I, CN, or SnR₃;
Q is H, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OH, OR, COR, OCOR, OSO₂R, SO₂R, SR; or Q together with the benzene ring to which it is attached is a fused ring system represented by structure A, B or C: 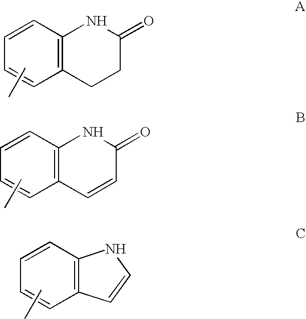
n is an integer of 1-4; and
m is an integer of 1-3.

28. The method according to claim 1, wherein said SARM compound is a compound represented by the structure of formula VI and/or its analog, derivative, isomer, metabolite, pharmaceutically acceptable salt, pharmaceutical product, hydrate, N-oxide, impurity, prodrug, polymorph, crystal, or any combination thereof

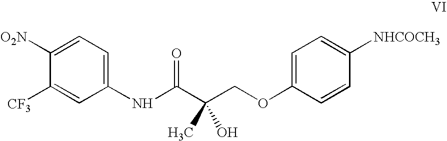

29. The method according to claim 1, wherein said SARM compound is a compound represented by the structure of formula VII I and/or its analog, derivative, isomer, metabolite, pharmaceutically acceptable salt, pharmaceutical product, hydrate, N-oxide, impurity, prodrug, polymorph, crystal, or any combination thereof

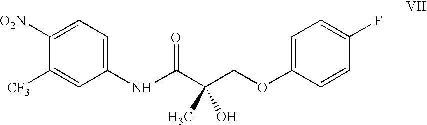

30. A method for detecting, identifying characterizing, purifying, obtaining structural information on, qualifying, screening, separating, fractionating and/or quantifying a Selective Androgen Receptor Modulator (SARM) compound in a sample, said method comprising the steps of:
obtaining said sample;
subjecting said sample to a chromatographic separation, thereby producing a chromatograph of said sample, said chromatograph comprising a series of peaks representing individual chemical compounds contained in said sample;
automatically collecting individual chemical compounds corresponding to peaks of said chromatograph into separate tubes;and
detecting the presence of said SARM compound in an aliquot from each tube;
thereby identifying said SARM compound in said sample.

31. The method according to claim 30, wherein said sample is a biological sample.

32. The method according to claim 30, wherein said sample is a blood serum sample, a plasma sample, a urine sample, a CSF sample, a saliva sample, a fecal sample, an isolated or precipitated fraction, a protein adduct, or a protein extract.

33. The method according to claim 30, wherein said detection step comprises subjecting each aliquot to mass spectroscopy (MS), MS-MS, UV, IR, NMR, fluorescence, radiochemical detection, electrochemical detection, chemiluminscent detection, evaporative light scatter detection (ESLD), hyphenated techniques or methods, or any combination thereof.

34. The method according to claim 30, wherein the detection step comprises measuring the UV absorbance of said SARM compound.

35. The method according to claim 30, wherein the detection step comprises measuring the molecular ion peak of said SARM compound by mass spectrometry (MS).

36. The method according to claim 35, wherein said mass spectrometry is negative ion mass spectrometry.

37. The method according to claim 30, further comprising the step of determining the concentration of said SARM in each aliquot by comparing the amount of obtained from said aliquot with a reference sample comprising known amounts of a reference SARM compound.

38. The method according to claim 30, wherein said chromatographic separation is by liquid chromatography (LC), High Performance Liquid Chromatography (HPLC), Thin Layer Chromatography (TLC), capillary electrophoresis (CE), microLC electrophoresis, nano LC electrophoresis, gel electrophoresis (GE), isoelectric focusing gel electrophoresis, or by sample concentration.

39. The method according to claim 30, wherein said chromatographic separation is by liquid chromatography (LC), and said detection step comprises measuring the molecular ion peak of said SARM compound by mass spectrometry (MS).

40. The method according to claim 30, wherein said chromatographic separation is by High Performance Liquid Chromatography (HPLC), and said detection step comprises measuring the UV absorbance of said SARM compound.

41. The method according to claim 30, wherein said chromatographic separation is by liquid chromatography (LC), and said detection step comprises measuring the molecular ion peak of said SARM compound by MS-MS.

42. The method according to claim 30, wherein said chromatographic separation is by capillary electrophoresis (CE), and said detection step comprises measuring the molecular ion peak of said SARM compound by mass spectrometry (MS).

43. The method according to claim 30, wherein said SARM compound is a compound represented by the structure of formula I and/or its analog, derivative, isomer, metabolite, pharmaceutically acceptable salt, pharmaceutical product, hydrate, N-oxide, impurity, prodrug, polymorph, crystal, or any combination thereof 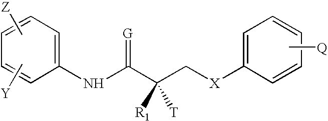
wherein G is O or S;
X is a bond, O, CH₂, NH, Se, PR, NO or NR;
T is OH, OR, —NHCOCH₃, or NHCOR
Z is NO₂, CN, COOH, COR, NHCOR or CONHR;
Y is CF₃, F, I, Br, Cl, CN, CR₃ or SnR₃;
Q is alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, SCN, NCS, OCN, NCO; or Q together with the benzene ring to which it is attached is a fused ring system represented by structure A, B or C: 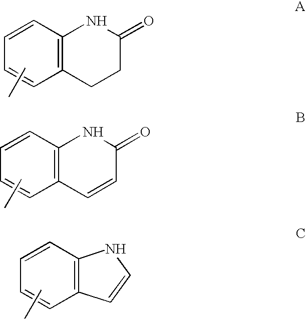
R is alkyl, haloalkyl, dihaloalkyl, trihaloalkyl, CH₂F, CHF₂, CF₃, CF₂CF₃, aryl, phenyl, halogen, alkenyl or OH; and
R₁ is CH₃, CH₂F, CHF₂, CF₃, CH₂CH₃, or CF₂CF₃.

44. The method according to claim 30, wherein said SARM compound is a compound represented by the structure of formula II and/or its analog, derivative, isomer, metabolite, pharmaceutically acceptable salt, pharmaceutical product, hydrate, N-oxide, impurity, prodrug, polymorph, crystal, or any combination thereof 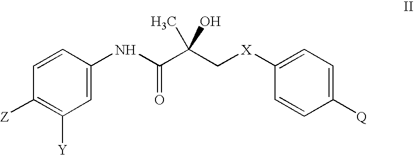
wherein X is a bond, O, CH₂, NH, Se, PR, NO or NR;
Z is NO₂, CN, COOH, COR, NHCOR or CONHR;
Y is CF₃, F, I, Br, Cl, CN, CR₃ or SnR₃;
Q is alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, SCN, NCS, OCN, NCO; or Q together with the benzene ring to which it is attached is a fused ring system represented by structure A, B or C: 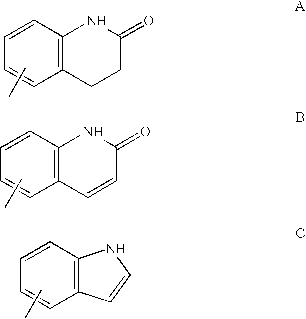
R is alkyl, haloalkyl, dihaloalkyl, trihaloalkyl, CH₂F, CHF₂, CF₃, CF₂CF₃, aryl, phenyl, halogen, alkenyl or OH.

45. The method according to claim 30, wherein said SARM compound is a compound represented by the structure of formula III and/or its analog, derivative, isomer, metabolite, pharmaceutically acceptable salt, pharmaceutical product, hydrate, N-oxide, impurity, prodrug, polymorph, crystal, or any combination thereof 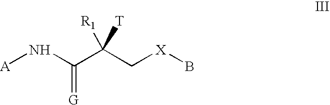
wherein X is a bond, O, CH₂, NH, Se, PR, NO or NR;
G is O or S;
R₁ is CH₃, CH₂F, CHF₂, CF₃, CH₂CH₃, or CF₂CF₃;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, halogen, alkenyl or OH;
A is a ring selected from: 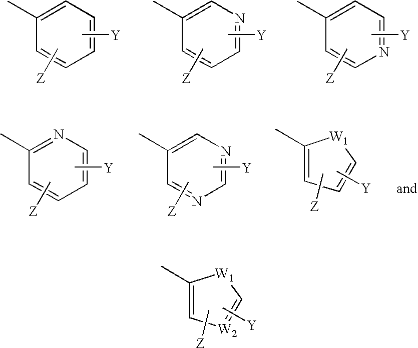
B is a ring selected from: 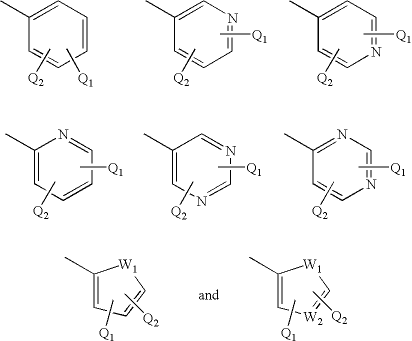
wherein A and B cannot simultaneously be a benzene ring;
Z is NO₂, CN, COOH, COR, NHCOR or CONHR;
Y is CF₃, F, I, Br, Cl, CN CR₃ or SnR₃;
Q₁ and Q₂ are independently of each other a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, SCN, NCS, OCN, NCO, 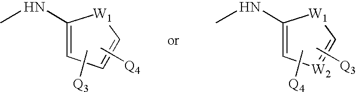
Q₃ and Q₄ are independently of each other a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R or SR, SCN, NCS, OCN, NCO;
W₁ is O, NH, NR, NO or S; and
W₂ is N or NO.

46. The method according to claim 30, wherein said SARM compound is a compound represented by the structure of formula IV and/or its analog, derivative, isomer, metabolite, pharmaceutically acceptable salt, pharmaceutical product, hydrate, N-oxide, impurity, prodrug, polymorph, crystal, or any combination thereof 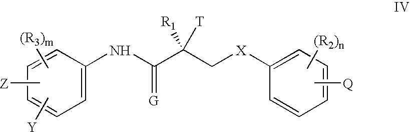
wherein X is a bond, O, CH₂, NH, Se, PR, NO or NR;
G is O or S;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
R₂ is F, Cl, Br, I, CH₃, CF₃, OH, CN, NO₂, NHCOCH₃, NHCOCF₃, NHCOR, alkyl, arylalkyl, OR, NH₂, NHR, NR₂, SR, SCN, NCS, OCN, NCO;
R₃ is F, Cl, Br, I, CN, NO₂, COR, COOH, CONHR, CF₃, SnR₃, or R₃ together with the benzene ring to which it is attached forms a fused ring system represented by the structure: 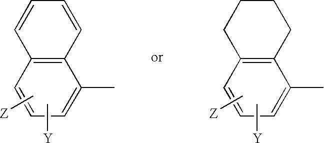
Z is NO₂, CN, COR, COOH, or CONHR;
Y is CF₃, F, Br, Cl, I, CN, or SnR₃;
Q is H, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OH, OR, COR, OCOR, OSO₂R, SO₂R, SR; or Q together with the benzene ring to which it is attached is a fused ring system represented by structure A, B or C: 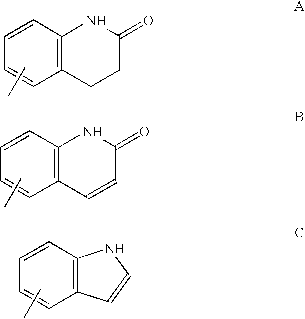
n is an integer of 1-4; and
m is an integer of 1-3.

47. The method according to claim 30, wherein said SARM compound is a compound represented by the structure of formula V and/or its analog, derivative, isomer, metabolite, pharmaceutically acceptable salt, pharmaceutical product, hydrate, N-oxide, impurity, prodrug, polymorph, crystal, or any combination thereof 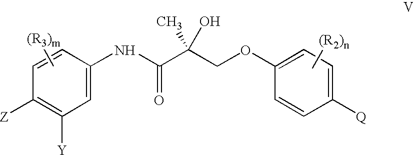
wherein
R₂ is F, Cl, Br, I, CH₃, CF₃, OH, CN, NO₂, NHCOCH₃, NHCOCF₃, NHCOR, alkyl, arylalkyl, OR, NH₂, NHR, NR₂, SR;
R₃ is F, Cl, Br, I, CN, NO₂, COR, COOH, CONHR, CF₃, SnR₃, or R₃ together with the benzene ring to which it is attached forms a fused ring system represented by the structure: 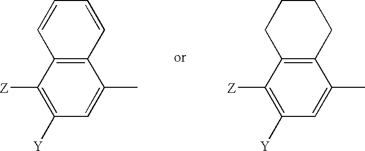
R is alkyl, haloalkyl, dihaloalkyl, trihaloalkyl, CH₂F, CHF₂, CF₃, CF₂CF₃, aryl, phenyl, halogen, alkenyl or OH;
Z is NO₂, CN, COR, COOH, or CONHR;
Y is CF₃ F, Br, Cl, I, CN, or SnR₃;
Q is H, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OH, OR, COR, OCOR, OSO₂R, SO₂R, SR; or Q together with the benzene ring to which it is attached is a fused ring system represented by structure A, B or C: 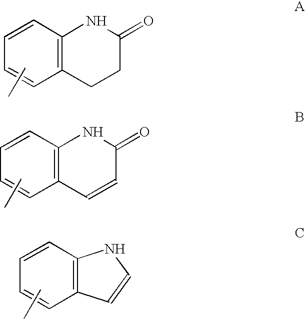
n is an integer of 1-4; and
m is an integer of 1-3.

48. The method according to claim 30, wherein said SARM compound is a compound represented by the structure of formula VI and/or its analog, derivative, isomer, metabolite, pharmaceutically acceptable salt, pharmaceutical product, hydrate, N-oxide, impurity, prodrug, polymorph, crystal, or any combination thereof

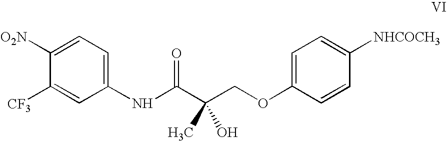

49. The method according to claim 30, wherein said SARM compound is a compound represented by the structure of formula VII I and/or its analog, derivative, isomer, metabolite, pharmaceutically acceptable salt, pharmaceutical product, hydrate, N-oxide, impurity, prodrug, polymorph, crystal, or any combination thereof [chemical structure]
